SEQUENCE LISTING (4 sequences)

SEQ ID NO: 1 - protein, 6 aa, Artificial Sequence
  Peptide 32230
FFVKLS

SEQ ID NO: 2 - protein, 16 aa, Artificial Sequence
  Peptide P28R
KKLDTFFVKLSLFTER

SEQ ID NO: 3 - protein, 17 aa, Homo sapiens
VFDEFKPLVEEPQNLIK

SEQ ID NO: 4 - protein, 16 aa, Artificial Sequence
  Peptide 32251
KKLDTFFPKLSLFTER

(19) United States
(12) Patent Application Publication
(10) Pub. No.: US 2021/0401926 A1
(43) Pub. Date: Dec. 30, 2021

(54) PHARMACEUTICAL FORMULATIONS OF PEPTIDE INHIBITORS

(71) Applicant: CANIMGUIDE THERAPEUTICS AB, Lund (SE)

(72) Inventors: Leif Håkansson, Höllviken (SE); Kristofer Ahlqvist, Höllviken (SE)

(21) Appl. No.: 17/273,951

(22) PCT Filed: Sep. 27, 2019
(86) PCT No.: PCT/US2019/053605

§ 371 Date: Mar. 5, 2021

Related U.S. Application Data
(60) Provisional application No. 62/738,859, filed on Sep. 28, 2018.

(51) Int. Cl. A61K 38/08; A61P 35/00

(52) U.S. Cl. CPC A61K 38/08; A61P 35/00

(57) ABSTRACT

Described herein are pharmaceutical products suitable for preparing pharmaceutical formulations comprising immunoregulatory peptide inhibitors. Described herein are pharmaceutical formulations comprising immunoregulatory peptide inhibitors. Also described herein are methods of ameliorating, inhibiting, reducing the symptoms of, or treating a cancer by administering a pharmaceutical formulation comprising an immunoregulatory peptide inhibitor.

### RELATED APPLICATIONS

This application claims the benefit of U.S. Provisional App. No. 62/738,859 filed Sep. 28, 2018, which is incorporated herein by reference in its entirety.

### REFERENCE TO SEQUENCE LISTING, TABLE, OR COMPUTER PROGRAM LISTING

The present application is being filed along with a Sequence Listing in electronic format. The Sequence Listing is provided as a file entitled CANIG009WOSEQLIST.TXT, created and last saved on Sep. 24, 2019, which is 1,109 bytes in size. The information in the electronic format of the Sequence Listing is incorporated herein by reference in its entirety.

### FIELD

Embodiments herein relate to pharmaceutical products and pharmaceutical formulations comprising immunoregulatory peptides, and methods of using the same.

### BACKGROUND

The immune system is finely tuned to detect and eradicate foreign molecules and, at the same time, avoid over reactivity, which could result in destruction of normal tissues resulting in autoimmune or chronic inflammatory diseases. The initiation of a specific immune response is a well-orchestrated chain of events culminating in the activation of effector functions, such as the release of cytokines, production of specific antibodies and/or cellular cytotoxic activity.

Although data indicate that the immune system is of major importance for cancer control (Dunn G P, et al., Immunity. 2004 21:137-48, Galon J, et al., Science. 2006 313:1960-4, Koebel C M, et al., Nature. 2007 450:903-7, Clinchy B, et al., Cancer. 2007 109:1742-9, Teng M W, et al., J Leukoc Biol. 2008 84:988-93) malignant tumors continue to grow and the efficacy of immunotherapy is rather poor with an objective remission rate of 10-20%. There can be several reasons for this apparent paradox, e.g., tumors avoid recognition by the immune system due to tumor antigens being weak self-antigens, poor antigen presentation due to down-regulation of TAP and MHC I and II) or induction of tolerance or cancer related immunosuppression. The impact of an hostile intra-tumoral milieu is demonstrated by results from animal experiments (Perdrizet G A, et al., J Exp Med. 1990; 171:1205-20, Yu P. et al., J Exp Med. 2005 201:779-91.) and human tumors (Gajewski T F, et al., J Immunother. 2006 29:233-40, Whiteside T L, Oncogene. 2008 27:5904-12).

Different types of immunosuppressor cells, regulatory T-cells, immature dendritic cells (iDC), tumor associated macrophages (TAM) and myeloid derived suppressor cells (MDSC), can function substantially in cancer related immunosuppression. The immune balance is generally skewed to a Th2 dominance characterized by cytokines, such as IL-4, IL-10 and PGE2. Additionally, other immunosuppressor mechanisms, such as serum blocking factors, circulating immune complexes, enhanced IL-1Ra production and enhanced intra-tumoral proteolytic activity can function in cancer related immunosuppression.

While investigating mechanisms for induction of interleukin-6 (IL-6) in cancer patients, immunoregulatory peptide sequences derived from serum albumin were found (see e.g., U.S. Pat. Nos. 7,960,126; 8,110,347; and 8,110,347; as well as, US Publication No. 2010/0323370, and PCT Pub. No. WO 2016/144650), each of which is hereby expressly incorporated by reference in their entireties. Interleukin-2 (IL-2) plays a major role in initiation and activation of the immune response and its capacity to induce lymphokine activated killer cells (LAK-cells), T-cell proliferation and cytotoxicity. Several reports have shown that peripheral blood mononuclear cells (PBMC) from cancer patients have a diminished capacity to both synthesize (Wanebo H J, et al., Cancer. 1986 57:656-62, Mantovani, G., et al., Diagn. Clin. Immunol. 1987 5: 104-111, Lauerova L, et al., Neoplasma 1999 46: 141-149) and respond to IL-2 (Tsubono M, et al., J Clin Lab Immunol 1990 33:107-115, Pellegrini P, et al., Cancer Immunol Immunother 1996 42:1-8). Soluble products from tumor explants or serum from cancer patients can inhibit cytokine production, inhibit IL-2 receptor expression (Botti C, et al., Intl J Biol Markers 1998 13:51-69, Lauerova L, et al., Neoplasma 1999 46:141-149) and/or reduce the proliferative capacity in normal T lymphocytes (Botti C, et al., Intl J Biol Markers 1998 13:51-69).

Integrins are a superfamily of transmembrane glycoproteins, found predominantly on leukocytes that mediate cell-cell and cell substratum interactions. Integrins play an important role in immune regulation, as well, in particular αLβ2, (Leukocyte Function Associated molecule-1, LFA-1) is of pivotal importance for the initiation and regulation of an immune response, tissue recruitment and migration of inflammatory cells and cytotoxic activity of lymphocytes (Hogg N, et al., J Cell Sci. 2003 116:4695-705, Giblin P A, et al., Curr Pharm Des. 2006 12:2771-95, Evans R, et al., Cell Sci. 2009 122:215-25). In addition, LFA-1 is involved in the proliferative response to interleukin-2 (Vyth-Dreese F A, Eur J Immunol. 1993 12:3292-9) and some fragments of albumin bind to LFA-1 and/or the IL-2 receptor thereby modulating the functional properties mediated through these receptors including immune cell proliferation (see U.S. Publication No. 2011/0262470, which is hereby expressly incorporated by reference in its entirety).

### SUMMARY

Option 1 comprises, consists essentially of, or consists of a pharmaceutical product comprising a first solution comprising an isolated peptide comprising amino acid sequence FFVKLS (SEQ ID NO: 1) dissolved in the first solution, the first solution having a pH less than 7 or about less than 7, and a sub-isotonic osmolarity. The pharmaceutical product can comprise a second solution comprising a tonicity agent and a base, in which the first and second solution generate an isotonic gel when combined with each other, and in which the isotonic gel comprises the isolated peptide at a concentration of at least 0.2 mg/ml or about at least 0.2 mg/ml and a pH of 6.5-7.5 or about 6.5-7.5.

Option 2 comprises, consists essentially of, or consists of the pharmaceutical product of option 1, in which the first solution is substantially free of particles greater than 0.2 μM in diameter or free of particles greater than about 0.2 μM in diameter, and wherein the second solution is substantially free of particles greater than 0.2 μM or greater than about 0.2 μM in diameter

Option 3 comprises, consists essentially of, or consists of the pharmaceutical product of any one of options 1-2, in which said first solution comprises less than or equal to 10 mM NaCl or less than or equal to about 10 mM NaCl but not zero.

Option 4 comprises, consists essentially of, or consists of the pharmaceutical product of any one of options 1-2, in which the first solution does not comprise NaCl.

Option 5 comprises, consists essentially of, or consists of the pharmaceutical product of any one of options 1-4, in which the first solution further comprises a buffer having a buffer capacity that is equivalent to 1.5 mM or about 1.5 mM sodium acetate or less, but not zero.

Option 6 comprises, consists essentially of, or consists of the pharmaceutical product of any one of options 1-4, in which the first solution further comprises sodium acetate at a concentration of less than or equal to 1.5 mM or less than or equal to about 1.5 mM or less but not zero.

Option 7 comprises, consists essentially of, or consists of the pharmaceutical product of any one of options 1-4, in which the first solution does not comprise buffer.

Option 8 comprises, consists essentially of, or consists of the pharmaceutical product of any one of options 1-7, in which the tonicity agent is NaCl, and in which the second solution is configured for the gel to comprise 100 mM-120 mM NaCl or about 100 mM-120 mM NaCl.

Option 9 comprises, consists essentially of, or consists of the pharmaceutical product of any one of options 1-8, in which the first solution is substantially free of gel.

Option 10 comprises, consists essentially of, or consists of the pharmaceutical product of any one of options 1-9, in which the isolated peptide dissolved in the first solution is substantially not in a beta-sheet conformation.

Option 11 comprises, consists essentially of, or consists of the pharmaceutical product of any one of options 1-11, in which the first solution is configured for the gel to comprise the isolated peptide at a concentration of at least 0.4 mg/ml or at least about 0.4 mg/ml.

Option 12 comprises, consists essentially of, or consists of the pharmaceutical product of any one of options 1-11, in which the isolated peptide comprises no more than 30 amino acid residues.

Option 13 comprises, consists essentially of, or consists of the pharmaceutical product of any one of options 1-12, in which the isolated peptide comprises the amino acid sequence KKLDTFFVKLSLFTER (SEQ ID NO: 2).

Option 14 comprises, consists essentially of, or consists of the pharmaceutical product of any one of options 1-11, in which the isolated peptide consists of the amino acid sequence KKLDTFFVKLSLFTER (SEQ ID NO: 2).

Option 15 comprises, consists essentially of, or consists of the pharmaceutical product of any one of options 1-11, in which the first solution is capable of maintaining at least 95% or at least about 95% of the isolated peptide dissolved in said first solution at 5° C. for at least 12-25 months or at least about 12-25 months.

Option 16 comprises, consists essentially of, or consists of the pharmaceutical product of any one of options 1-15, in which the first solution and the second solution are configured for the gel to comprise: at least 0.4 mg/ml or at least about 0.4 mg/ml of the isolated peptide consisting of the amino acid sequence KKLDTFFVKLSLFTER (SEQ ID NO: 2); 30-40 mM or at least about 30-40 mM acetic acid; 1.2-1.6 mM or at least about 1.2-1.6 mM sodium acetate; less than or equal to 30 mM or less than or equal to about 30 mM sodium hydroxide; and 100-120 mM or about 100-120 mM sodium chloride. The gel can have an osmolarity of 280-300 mOSmol/L or about 280-300 mOSmol/L.

Option 17 comprises, consists essentially of, or consists of method of manufacturing the pharmaceutical product of any one of options 1-16. The method can comprise sterile-filtering a precursor solution comprising an isolated peptide comprising amino acid sequence FFVKLS (SEQ ID NO: 1) dissolved in the precursor solution at a concentration of at least or at least about 0.2 mg/ml, wherein the precursor solution has a pH of less than or less than about 7 and a sub-isotonic osmolarity, producing said first solution. The method can comprise providing the second solution comprising the tonicity agent and the base.

Option 18 comprises, consists essentially of, or consists of the method of option 17, in which the precursor solution is sterile-filtered with a filter of pore size of about 0.2 μM or 0.2 μM.

Option 19 comprises, consists essentially of, or consists of the method of any one of options 17-18, in which the precursor solution has a pH less than or less than about 4.5.

Option 20 comprises, consists essentially of, or consists of the method of any one of options 17-19, in which the isolated peptide comprises no more than 30 amino acid residues.

Option 21 comprises, consists essentially of, or consists of the method of any one of options 17-20, in which the isolated peptide comprises the amino acid sequence KKLDTFFVKLSLFTER (SEQ ID NO: 2).

Option 22 comprises, consists essentially of, or consists of the method of any one of options 17-21, in which the isolated peptide consists of the amino acid sequence KKLDTFFVKLSLFTER (SEQ ID NO: 2).

Option 23 comprises, consists essentially of, or consists of a method of preparing a pharmaceutical formulation from the pharmaceutical product of any one of options 1-22. The method can comprise combining the first solution and the second solution to form said gel.

Option 24 comprises, consists essentially of, or consists of a pharmaceutical formulation comprising a gel. The gel can comprise at least or at least about 0.4 mg/ml of an isolated peptide comprising amino acid sequence FFVKLS (SEQ ID NO: 1). The gel can comprise a buffer system comprising acetic acid and sodium acetate, the buffer system comprising less than or equal to 1.6 mM or less than or equal to about 1.6 mM sodium acetate. The gel can comprise a tonicity agent. The gel can be isotonic and have a pH of 4.5-7.5 or about 4.5-7.5.

Option 25 comprises, consists essentially of, or consists of the pharmaceutical formulation of option 24, in which the gel is substantially free of particles greater than or greater than about 0.2 μM in diameter.

Option 26 comprises, consists essentially of, or consists of the pharmaceutical formulation of any one of options 25-26, in which the tonicity agent is sodium chloride at a concentration of 100-120 mM or about 100-120 mM.

Option 27 comprises, consists essentially of, or consists of the pharmaceutical formulation of any one of options 24-26, in which the buffer system comprises 30-40 mM or about 30-40 mM acetic acid and 1.2-1.6 mM or about 1.2-1.6 mM sodium acetate.

Option 28 comprises, consists essentially of, or consists of the pharmaceutical formulation of any one of options 24-27, further comprising sodium hydroxide at a concentration of less than or equal to 30 mM or less than or equal to about 30 mM but not zero.

Option 29 comprises, consists essentially of, or consists of the pharmaceutical formulation of any one of options 24-28, in which the gel has an osmolarity of 280-300 mOSmol/L or about 280-300 mOSmol/L.

Option 30 comprises, consists essentially of, or consists of the pharmaceutical formulation of any one of options 24-29, in which the isolated peptide comprises no more than 30 amino acid residues.

Option 31 comprises, consists essentially of, or consists of the pharmaceutical formulation of any one of options 24-30, in which the isolated peptide comprises the amino acid sequence KKLDTFFVKLSLFTER (SEQ ID NO: 2).

Option 32 comprises, consists essentially of, or consists of the pharmaceutical formulation of any one of options 24-31, in which the isolated peptide consists of the amino acid sequence KKLDTFFVKLSLFTER (SEQ ID NO: 2).

Option 33 comprises, consists essentially of, or consists of a pharmaceutical formulation. The pharmaceutical formulation can comprise an isolated peptide comprising amino acid sequence FFVKLS (SEQ ID NO: 1), wherein the isolated peptide is dissolved in the pharmaceutical formulation at a concentration of 0.2-20 mg/ml, or at about 0.2-20 mg/ml. The pharmaceutical formulation can comprise a non-ionic tonicity agent. The pharmaceutical formulation can be isotonic, and have a pH of 5.0-5.5 or about 5.0-5.5, and the pharmaceutical formulation can be a liquid.

Option 34 comprises, consists essentially of, or consists of the pharmaceutical formulation of option 33, in which the pharmaceutical composition is substantially free of particles having a diameter greater than or greater than about 0.2 μM.

Option 35 comprises, consists essentially of, or consists of the pharmaceutical formulation of any one of options 33-34, further comprising a weak acid. The peptide and the weak acid can comprise a buffer system that maintains the pharmaceutical formulation at a pH of 5.0-5.5 or about 5.0-5.5.

Option 36 comprises, consists essentially of, or consists of the pharmaceutical formulation of option 35, in which the weak acid is acetic acid, which is present at a concentration of 0.01M or about 0.01M.

Option 37 comprises, consists essentially of, or consists of the pharmaceutical formulation of any one of options 33-36, in which the non-ionic tonicity agent is glucose, which is present at a concentration of 0.2 M-0.4 M or about 0.2M-0.4M.

Option 38 comprises, consists essentially of, or consists of the pharmaceutical formulation of any one of options 33-37, in which the isolated peptide is at a concentration of 0.2-5 mg/ml, about 0.2-5 mg/ml, 0.2-10 mg/ml, about 0.2-10 mg/ml, 1-5 mg/ml, about 1-5 mg/ml, 1-10 mg/ml, or about 1-10 mg/ml.

Option 39 comprises, consists essentially of, or consists of the pharmaceutical formulation of any one of options 33-38, in which the isolated peptide comprises no more than 30 amino acid residues.

Option 40 comprises, consists essentially of, or consists of the pharmaceutical formulation of any one of options 33-39, in which the isolated peptide comprises the amino acid sequence KKLDTFFVKLSLFTER (SEQ ID NO: 2).

Option 41 comprises, consists essentially of, or consists of the pharmaceutical formulation of any one of options 33-38, in which the isolated peptide consists of the amino acid sequence KKLDTFFVKLSLFTER (SEQ ID NO: 2).

Option 42 comprises, consists essentially of, or consists of the pharmaceutical formulation of any one of options 33-41, further comprising 0.01 M or about 0.01M acetic acid. The isolated peptide can consist of the amino acid sequence KKLDTFFVKLSLFTER (SEQ ID NO: 2) and the isolated peptide can present at a concentration of 1-10 mg/ml or about 1-10 mg/ml. The non-ionic tonicity agent can be glucose, which is present at a concentration of 0.2-0.4M or about 0.2-0.4 M.

Option 43 comprises, consists essentially of, or consists of a method of ameliorating, inhibiting, reducing the symptoms of, or treating a cancer in a patient in need thereof, the method comprising administering an effective amount of the pharmaceutical formulation of any one of options 24-42 of the patient.

Option 44 comprises, consists essentially of, or consists of the method of option 43, wherein the effective amount is up to 1 mg/kg or up to 5 mg/kg.

Option 45 comprises, consists essentially of, or consists of the method of any one of options 43-44, in which the effective amount of the pharmaceutical formulation comprises 8-800 μg or about 8-800 μg of the isolated peptide.

Option 46 comprises, consists essentially of, or consists of the method of any one of options 43-44, in which the effective amount of the pharmaceutical formulation comprises 60-100 μg or about 60-100 μg of the isolated peptide.

Option 47 comprises, consists essentially of, or consists of the method of any one of options 43-46, wherein the pharmaceutical formulation is administered intratumorally, subcutaneously, lymphatically, and/or to an interstitial fluid of the patient.

Option 48 comprises, consists essentially of, or consists of the method of any one of options 43-47, further comprising repeating the administration of the pharmaceutical formulation.

Option 49 comprises, consists essentially of, or consists of the method of any one of options 43-48, in which the cancer comprises a tumor.

Option 50 comprises, consists essentially of, or consists of the method of any one of options 43-49, in which the cancer is selected from the group consisting of: head and neck cancer, breast cancer, renal cancer, colorectal cancer, skin cancer, ovarian cancer, prostate cancer, pancreatic cancer, lung cancer, malignant melanoma, small cell lung cancer, non-small lung cancer (adenocarcinoma), squamous cell carcinoma, bladder cancer, osteosarcoma, bronchial cancer, or hematopoietic cell cancer

Option 51 comprises, consists essentially of, or consists of the method of any one of options 43-50, further comprising receiving results of detection of a presence and/or level of peptide P3028 (SEQ ID NO: 3) or a P3028 structure in a sample of the patient, such as a sample comprising hematopoietic tissue, a body fluid, a blood sample, a tumor biopsy, or a biopsy of tissue surrounding the tumor. In some embodiments, the sample comprises hematopoietic tissue, a blood sample, or a tumor biopsy.

Option 52 comprises, consists essentially of, or consists of the method of option 51, further comprising selecting the patient for receiving the effective amount of the pharmaceutical formulation if a denatured or damaged albumin, such as a P3028 structure, is present, or exceeds a predetermined level in the sample.

Option 53 comprises, consists essentially of, or consists of the method of any one of options 43-52, further comprising receiving results of detection of a presence of immune cells in a sample of the patient, such as a tumor biopsy.

Option 54 comprises, consists essentially of, or consists of the method of option 53, wherein the sample of the patient is collected at least 5 days after the administration of the pharmaceutical compositions.

Option 55 comprises, consists essentially of, or consists of the method of any one of options 53-54, further comprising selecting the patient for receiving the effective amount of the pharmaceutical formulation if the immune cells are present in the sample.

Option 56 comprises, consists essentially of, or consists of the method of any one of options 43-55, further comprising selecting the patient as comprising peptide 3028 and/or immune cells in a sample of the patient, such as a sample comprising hematopoietic tissue, a body fluid, a blood sample, or a tumor biopsy. In some embodiments, the sample comprises hematopoietic tissue, a blood sample, or a tumor biopsy.

Option 57 comprises, consists essentially of, or consists of the method of any one of options 43-56, further comprising selecting the patient as comprising a tumor that is substantially free of T cell infiltrates, a tumor that comprises a majority of T-cell infiltrates in the stroma, or a tumor that is inflamed and infiltrated by inactive T cells.

Option 58 comprises, consists essentially of, or consists of the method of any one of options 43-57, further comprising detecting an inflammatory response to the tumor after administration of the pharmaceutical formulation, such as effector cell and/or suppressor cell infiltration of the tumor.

Option 59 comprises, consists essentially of, or consists of the method of any one of options 43-58, further comprising detecting death of tumor cells, such as apoptosis or necrosis, after administration of the pharmaceutical composition.

Option 61 comprises, consists essentially of, or consists of the method of any one of option 43-59, further comprising administering an additional therapeutic agent to the patient.

Option 62 comprises, consists essentially of, or consists of the method of option 61, wherein the additional therapeutic agent comprises an antibody that specifically binds to PD-1 or PDL-1, or that is bispecific for PD-1 and PDL-1.

### BRIEF DESCRIPTION OF THE DRAWINGS

FIGS. 1A-D are as series of graphs illustrating stimulatory activity of P28R on a suppressed proliferative response to IL-2. FIGS. 1A, 1B, 1C, and 1D respectively illustrate stimulatory activity for four different cancer patients.

FIG. 2A-B are a series of graphs illustrating effects of the full length peptide P28R and the 6 amino acid central sequence (32230, FFVKLS, SEQ ID NO: 1) in culture medium containing normal human AB serum. Activation is determined as percentage of cells with enhanced marker CD69 or CD71 using flow cytometry. PBMCs were incubated with the peptides (40 μg/mL) for 24 hours in RPMI plus 10% human AB serum. FIG. 2A illustrates the results of two experiments (420 and 422) performed for each peptide. FIG. 2B illustrates the results of two experiments (424 and 426) performed for each peptide.

FIG. 3 is a graph illustrating a comparison of the full length peptide P28R and the 6 amino acid “P28 core” sequence (32230, FFVKLS, SEQ ID NO: 1) in culture medium containing sera from two different cancer patients (“human ca serum 1” 430 and (“human ca serum 2” 432).

FIG. 4 is a graph showing evaluation of P28R treatment in 7 dogs with breast tumours compared with 5 untreated control dogs, in accordance with some embodiments herein. In representative pictures (n=1-5), the total number of tumour cells from treated (dark bars 881 in P28R #4, #7, #8, #11, #13, #16, and #17) and from control tumours (dark bars 882 in Controls #2, #3, #4, #5, and #6) was counted and compared with the number of inflammatory cells (light grey bars 883).

FIGS. 5A-B are a series of graphs showing that subcutaneous P28R treatment (single dose 80 microgram) of spontaneous canine breast tumours results in recruitment of inflammatory cells and tumour cell death. (Control N=12 and treated N=5).

FIGS. 6A-C are a series of microscope images of three different tongue cancers double stained using antibodies directed against P3028 (red) and CD3 (brown). The immune desert cancer (FIG. 6A) has a strong expression of 3028 and only few scattered T-cells in the stroma. The immune excluded cancer in the middle (FIG. 6B) has a strong expression of 3028 and T-cells infiltrating in the stroma, the inflamed cancer to the right (FIG. 6C) is only faintly stained for 3028 and has a very strong infiltration of T-cells.

FIGS. 7A-B are a series of microscope images of tumour section from a breast cancer patient showing inflammatory cells stained by an antibody directed against CD11a. Fresh frozen tumour sections without any fixation were incubated with buffer (FIG. 7A) or P28R (FIG. 7B) before staining.

### DETAILED DESCRIPTION

Described herein are pharmaceutical products that comprise, consist essentially of, or consist of peptide inhibitors, as well as pharmaceutical formulations of peptide inhibitors, and methods of making and using the pharmaceutical products and formulations. The peptide inhibitors interact with immunoregulatory peptides that cause immunosuppression in a human (e.g., a human having cancer), and have been show to alleviate immunosuppression of immune cells in cancer patient serum in vitro, and cause regressive changes and eradication of mammalian tumors in vivo (See Examples 1-4 and 8). These results were further confirmed in additional dose escalation studies in dogs (See Example 4). It has been observed herein that conventional formulations of peptide inhibitors can affect the health of cells near the site of injection, for example due to low osmolarity and/or the presence of acetate (See Example 9). Moreover, it has been observed herein that peptide inhibitors can form a gel at high pH and/or in the presence of elevated sodium, which can interfere with sterile filtration by clogging the pores of a filter (See Examples 10 and 11). Accordingly, described in accordance with some embodiments herein are pharmaceutical products, pharmaceutical formulations, that are sterile, stably maintain peptide inhibitors, and permit administration of suitable dosages of peptide inhibitors while avoiding adverse effects associated with acetate and non-isotonic osmolarity.

In some embodiments, a pharmaceutical product comprising a first solution comprising a peptide inhibitor such as P28R at an acidic pH, and a second solution comprising a base is provided. The first and second solutions can be combined prior to use in order to form a gel comprising the peptide inhibitor, at a pH and an osmolality suitable for administration to a cancer patient. The first solution can be sterile, for example via sterile filtering (the acidic pH and low sodium content of the first solution can avoid the formation of a gel that would clog the pores of a filter; See Example 10). The first solution can have an acidic pH and a sub-isotonic osmolarity. The second solution can have a basic pH and an above-isotonic osmolarity. In some embodiments, the peptide inhibitor is a peptide comprising, consisting essentially of, or consisting of the amino acid sequence FFVKLS (SEQ ID NO: 1). The peptide can comprise no more than 50, 45, 40, 35, 30, 25, or 20 amino acids. In some embodiments, the peptide inhibitor is a peptide comprising, consisting essentially of, or consisting of the amino acid sequence KKLDTFFVKLSLFTER (SEQ ID NO: 2).

It has been observed in dose escalation studies described herein that doubling and quadrupling the initial 80 microgram dose of P28R maintained anti-tumor effects, without observed adverse effects (See Example 4). Accordingly, it is contemplated that formulations comprising even higher concentration of peptide inhibitors can be safe and effective. Described in accordance with some embodiments herein are pharmaceutical products comprising, consisting of, consisting essentially of, or configured to be constituted into pharmaceutical formulations of peptide inhibitors at a concentration of about 0.2-20 mg/ml, for example, 0.2-5 mg/ml, 0.2-10 mg/ml, 1-5 mg/ml, 1-10 mg/ml, or 1-20 mg/ml. The pharmaceutical formulations can comprise a peptide inhibitor as described herein, and a non-ionic tonicity agent. The formulation can be isotonic, and have a pH of 5.0-5.5 or about 5.0-5.5. The formulation can be a liquid. The peptide inhibitor can remain dissolved in the liquid. As such, the pharmaceutical formulation can be free, or substantially free of gel. As such, the pharmaceutical formulation can be free, or substantially free of precipitates.

As used herein, “substantially free” has its ordinary and customary meaning as would be understood by one of ordinary skill in the art in view of this disclosure. It refers to trace amounts that would have no appreciable effects on the stability and efficacy of the pharmaceutical product or formulation, and also encompasses an absence of the specified substance. If additional numerical precision is of interest, in some embodiments, a composition (such as a pharmaceutical product or pharmaceutical formulation) is substantially free of a substance when it contains no more than 5% (w/w) of the substance, for example, no more than 5%, 4%, 3%, 2%, 1%, 0.9%, 0.5%, 0.2%, 0.1%, 0.05%, or 0.01%, including ranges between any two of the listed values.

Also described herein are methods of ameliorating, inhibiting, reducing the symptoms of, or treating a cancer in a patient in need thereof, the method comprising administering an effective amount of a pharmaceutical formulation as described herein to the patient.

### Peptide Inhibitors

Some embodiments include peptide inhibitors. It has been shown that the albumin-derived peptide P3028 (VFDEFKPLVEEPQNLIK—SEQ ID NO: 3) is sufficient to inhibit immune cell proliferation and activation, and impose a blockade the immune system, as do “P3028 structures” (e.g., damaged and/or denatured albumin, which may be identified by antibodies and/or peptides specific for P3028)(See Example 1; See also U.S. Pat. No. 9,796,77, and PCT Pub. Nos. WO 2015/035332 and WO 2016/144650, each of which is incorporated by reference in its entirety herein). Peptide-based binding partners of P3028 were developed. It was shown that peptides comprising, consisting essentially of, and consisting of the amino acid sequence FFVKLS (SEQ ID NO: 1), for example P28R (KKLDTFFVKLSLFTER: SEQ ID NO: 2) are sufficient to bind to peptide P3028 (See PCT Pub. No. WO 2016/144650 at Examples 10 and 36). Moreover, peptide inhibitors comprising, consisting essentially of, and consisting of the motif FFVKLS (SEQ ID NO: 1), for example P28R (SEQ ID NO: 2) are sufficient to alleviate the immunosuppressive effects of P3028 (See Examples 1-2), and can alleviate immunosuppression, causing immune system infiltration and destruction of tumors in vivo (See Examples 3-5).

The peptide inhibitors of compositions, pharmaceutical products, pharmaceutical formulations, and methods of some embodiments can bind to and inhibit immunoregulatory peptides such as P3028 and/or one or more other albumin-derived immunoregulatory peptides (See, e.g., blocker peptides identified in Tables 1-4 of PCT Pub. No. WO 2016/144650). The peptide inhibitors of some embodiments can include, but are not limited to: peptides, cyclic peptides, peptidomimetics, and proteins, including, for example, synthetic peptides. The following section provides more details on antibody or antibody fragment-based peptide inhibitors.

In compositions, pharmaceutical products, pharmaceutical formulations, and methods of some embodiments, the peptide inhibitor comprises, consists essentially of, or consists of the amino acid sequence FFVKLS (SEQ ID NO: 1). In compositions, pharmaceutical products, pharmaceutical formulations, and methods of some embodiments, the peptide inhibitor comprises the amino acid sequence FFVKLS (SEQ ID NO: 1), and has a length of no more than 100 amino acids, for example, no more than 100, 90, 80, 70, 60, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, or 6 amino acid residues, including ranges between any two of the listed values, for example, 6-100, 6-50, 6-30, 6-29, 6-25, 6-20, 6-15, 6-16, 10-100, 10-50, 10-30, 10-29, 10-25, 10-20, 10-16, 15-100, 15-50, 15-30, 15-29, 15-25, 15-20, or 15-16 amino acid residues. In compositions, pharmaceutical products, formulations, and methods of some embodiments, the peptide inhibitor comprises, consists essentially of, or consists of P28R (KKLDTFFVKLSLFTER; SEQ ID NO: 2).

The peptide inhibitors of some embodiments bind to a peptide comprising, consisting essentially of, or consisting of the amino acid sequence of P3028 (VFDEFKPLVEEPQNLIK—SEQ ID NO: 3). The peptide inhibitors of some embodiments inhibit binding of P3028 to the LFA-1 receptor, thus de-blocking the LFA-1 receptor. Accordingly, the peptide inhibitors of some embodiments are sufficient to induce activation of immune cells that have been immunosuppressed by damaged or denatured albumin or albumin peptides such as P3028 structures. Examples of immune cell activation include, but are not limited to proliferation, enhanced expression of CD69 and/or CD71, secretion of IL-12 or IFNγ, or secretion of perforin or granzyme B, enhanced cytotoxicity, cell migration, or cytokine production, or two or more of the listed items. In some embodiments, a pharmaceutical product, pharmaceutical formulation, composition, or method comprises an amount of peptide inhibitor effective to inhibit binding of P3028 to the LFA-1 receptor, thus de-blocking the LFA-1 receptor. In some embodiments, a pharmaceutical product, pharmaceutical formulation, composition, or method comprises an amount of peptide inhibitor effective to activation of immune cells that have been immunosuppressed by damaged or denatured albumin or albumin peptides such as P3028 structures.

Unless explicitly started otherwise, whenever an “isolated peptide” is mentioned herein, for example in the context of a pharmaceutical product, formulation, composition, or method of some embodiments, unless stated otherwise, it will be understood to refer to a “peptide inhibitor” as described herein.

### Buffer Systems

“Buffer” and “buffer system” as used herein have their ordinary and customary meaning as would be understood by one of ordinary skill in the art in view of this disclosure. They refer to compositions that resist changes in pH in a solution. Thus, buffers can facilitate the maintenance of a composition, pharmaceutical product, and/or pharmaceutical formulation of some embodiments within a specified pH range.

A buffer or buffer system can comprise, consist essentially of, or consist of a weak acid and its conjugate base. Kₐ refers to the dissociation constant of the proton of an acid and can be calculated as Kₐ=([H⁺][A⁻]/[HA]). Buffering capacity is maximal at the pKₐ of the buffer (i.e., the negative log of Kₐ). As such, buffers can be selected to have a pKₐ that is at or near the desired pH or pH range of the substance that is being buffered, for example, a pKₐ within ±2 of the desired pH, a pKₐ within ±1 of the desired pH, or a pKₐ within ±0.5 of the desired pH. It is noted that some acids can have more than one proton, and as such, a buffer system can have more than one pKₐ. In addition to conventional small molecule buffers, such as phosphate, acetate, citrate, and borate systems, amino acids represent weak acids, as do acidic side chains of amino acids, and thus these amino acids and side chain can also provide buffering. Accordingly, it is contemplated that acidic side chains (such as Asp, Glu, and His) of some embodiments can contribute to buffering of peptide inhibitors as described herein, especially at pH ranges at or near the pKₐ's of the acidic side chains (is noted that the pKₐ's of the side chains of Asp, Glu, and His are 3.7, 4.3, and 6.5, respectively).

“Buffer capacity” has its ordinary and customary meaning as would be understood by one of ordinary skill in the art in view of this disclosure. It refers to the amount of strong acid or strong base that is needed to change the pH by one unit. Typically, buffer capacity units are expressed as gram or molar equivalents. Buffer capacity can be determined empirically (for example by acid and/or base titration), and can also be calculated. By way of example, Equation (I), below, can be used to estimate the buffer capacity for a buffer system comprising an acid [HA] and its conjugate base [A-], and having a dissociation constant of Kₐ:

Buffer capacity=[A⁻]+([H⁺][A⁻]/Kₐ) (I)

It will be appreciated that comparisons of buffer capacity (for example, between a buffer and a reference buffer such as sodium acetate or sodium citrate) are suitably made under comparable conditions, such as temperature and/or pressure. In some embodiments, buffer capacities are determined at or near room temperature at 1 atm of pressure. In some embodiments, buffer capacities are determined at standard temperature and pressure (0° C. and 1 atm).

In some embodiments, a composition, pharmaceutical product, or formulation comprises a buffer system selected from the group consisting of: Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO, TES, and acidic side chains of a peptide inhibitor as described herein, including two or more of the listed items. In some embodiments, a composition, pharmaceutical product, or pharmaceutical formulation comprises a buffer system selected from the group consisting of acetate and acidic side chains of a peptide inhibitor as described herein, or both of the listed items. In some embodiments, a composition, pharmaceutical product, or pharmaceutical formulation comprises a buffer system comprising acetate and acidic side chains of a peptide inhibitor as described herein.

### Tonicity Agents

In order for a composition, pharmaceutical product, or formulation of some embodiments to have a suitable tonicity (so as to minimize pain and tissue damage upon administration of the composition, pharmaceutical product, or formulation), the composition, pharmaceutical product, or pharmaceutical formulation may comprise a tonicity agent. A tonicity agent can be included in a composition, pharmaceutical product, or formulation as described herein so as to bring the osmolality of the composition, pharmaceutical product, or pharmaceutical formulation at or near physiological ranges. Typically, the osmolarity of human blood is 275-299 mOsm/L or about 275-299 mOsm/L, for example, 275-295 mOsm/L, 275-296 mOsm/L, 275-297 mOsm/L, 280-295 mOsm/L, 280-296 mOsm/L, 280-297 mOsm/L, 281-295 mOsm/L, 281-296 mOsm/kg, or 281-297 mOsm/L.

While a variety of factors (such as dehydration) can affect the osmolarity of the blood of an individual human, it will be understood that in accordance with embodiments herein, an isotonic osmolality refers to an osmolality at or near the osmolarity of the patient's blood, or within the expected osmolarity range of a patient's blood, so as to permit administration thereto while inhibiting discomfort and adverse physiological effects due to differences in osmolality. If additional numerical precision is of interest, in some embodiments, isotonic osmolarity is within ±10% of the osomolarity of a patient's blood, for example within ±10%, ±9%, ±8%, ±7%, ±6%, ±5%, ±4%, ±3%, ±2%, or ±1%, including ranges between any two of the listed values. In some embodiments, an isotonic osmolarity refers to about 280-300 mOsmol/L, or 280-300 mOsmol/L. As such, it will also be understood that a “sub-isotonic” osmolarity refers to an osmolarity that is numerically below an isotonic osmolarity, for example, less than 280 mOsm/L, 270 mOsm/L, or 260 mOsm/L. It will also be understood that an osmolarity greater than tonic refers to an osmolarity that is numerically greater than an isotonic osmolarity, for example, greater than 300 mOsm/L, 310 mOsM/L, or 320 mOsm/L.

In some embodiments, a composition, pharmaceutical product, or formulation has an osomolarity of about 280-300 mOsmol/L, or 280-300 mOsmol/L. In some embodiments, a composition, pharmaceutical product, or formulation has an osmolarity of 270-280 mOsmol/L, 270-290 mOsmol/L, 270-300 mOsmol/L, 270-310 mOsmol/L, 280-290 mOsmol/L, 280-300 mOsmol/L, 280-310 mOsmol/L, 290-300 mOsmol/L, 290-310 mOsmol/L, or 300-310 mOsmol/L.

Examples of suitable tonicity agents for compositions, pharmaceutical products, or formulations of some embodiments include, but are not limited to, sodium chloride, potassium chloride, glucose, sucrose, dextrose, mannitol, sorbitol, trehalose, glycerol, or combinations or two or more of these. Examples of tonicity agents can also be found in Remington's The Science and Practice of Pharmacy, 21st Ed., Lippincott Williams & Wilkins (2005), and Gilman et al. (Eds.) (1990); Goodman and Gilman's: The Pharmacological Basis of Therapeutics, 8th Ed., Pergamon Press, each of which is incorporated by reference in its entirety herein.

In some embodiments, the composition, pharmaceutical product, or formulation (for example a formulation comprising at least 10 mg/ml of peptide inhibitor, such as Formulation C) comprises a non-ionic tonicity agent, such as glucose, sucrose, dextrose, mannitol, glycerin, or combinations of two or more of the listed items. In some embodiments, the tonicity agent of the composition, pharmaceutical product, or formulation does not comprise sodium. As such, in some embodiments, the composition, pharmaceutical product, or formulation is free or substantially free of sodium. In some embodiments, the tonicity agent of the composition, pharmaceutical product, or formulation does not comprise sodium chloride. As such, in some embodiments, the composition, pharmaceutical product, or formulation is free or substantially free of sodium chloride.

### Carriers

The composition, pharmaceutical product, or pharmaceutical formulation can be formulated in a suitable carrier. The carrier can be a solvent for some or all of the components of the composition, pharmaceutical product, or pharmaceutical formulation. Examples of suitable carriers include, but are not limited to syrups, elixirs, emulsions and/or suspensions, for example comprising, consisting essentially of, or consisting include ethanol, glycerol, propylene glycol, polyethylene glycol, liquid sucrose, sorbitol, and/or water. In some embodiments, the composition, pharmaceutical product, or formulation is an aqueous solution. In some embodiments, the composition, pharmaceutical product, or pharmaceutical formulation is formulated in a carrier that comprises, consists essentially of, or consists of water. In some embodiments, the composition, pharmaceutical product, or pharmaceutical formulation is formulated in a carrier that is water. In some embodiments, the composition, pharmaceutical product, or pharmaceutical formulation comprises, consists essentially of, or consists of an aqueous formulation.

### Pharmaceutical Products

In some embodiments, a pharmaceutical product comprises, consists essentially of, or consists of a formulation of a peptide inhibitor, or of components of a formulation of a peptide inhibitor. The pharmaceutical product of some embodiments comprises a first solution comprising the peptide inhibitor at a pH of less than 7 or about less than 7 (for example, less than or equal to 7, 6, 5, 4.5, 4, 3.5, or 3), and having a sub-isotonic osmolality. The first solution can be sterile, for example, by sterile filtration. It has been shown herein, that at relatively high pH (such as a pH of 4.5 or greater), the peptide inhibitor can form a gel that interferes with sterile filtration, and can precipitate out of solution (See Examples 10-11). Accordingly, it is contemplated that the specified acidic pH's can be advantageous for maintaining the peptide inhibitor in a sterile solution that can be filtered. The pharmaceutical product can further comprise a second solution comprising a tonicity agent. The first and second solution, when combined, can produce an isotonic gel comprising the peptide inhibitor at a concentration of at least 0.2 mg/ml or about at least 0.2 mg/ml and a pH of 6-8 or about 6-8. The peptide inhibitor can comprise, consist essentially of, or consist of P28 core (FFVKLS; SEQ ID NO: 1). In some embodiments, the first solution is substantially free of particles greater than 0.2 μM or greater than about 0.2 μM in diameter. The second solution can be substantially free of particles greater than 0.2 μM or greater than about 0.2 μM in diameter. In some embodiments, the first and second solution, when combined, produce an isotonic gel having a pH of 4.5-7.5, about 4.5-7.5, 6.5-7.5, or about 6.5-7.5. In some embodiments, the first and second solution, when combined, produce an isotonic gel comprising the peptide inhibitor at a concentration of at least 0.2 mg/ml or at least about 0.2 mg/ml and a pH of 4.5-7.5 or about 4.5-7.5. In some embodiments, the first and second solution, when combined, produce an isotonic gel comprising the peptide inhibitor at a concentration of at least 0.2 mg/ml or about at least 0.2 mg/ml and a pH of 6.5-7.5 or about 6.5-7.5.

It has been shown herein that increasing concentrations of NaCl can cause peptide inhibitors as described herein to aggregate and form a gel (Example 11). The gel can interfere with sterile filtration. Accordingly, in some embodiments, the peptide inhibitor is in a liquid such as the first solution. The first solution can be sterile, for example by way of sterile filtration through a filter having a pore size of no more than 0.2 μM, for example, 0.2 μM or less, or 0.1 μM or less. As such, the gel formed by the combination of the first and second solution can be substantially free of particles having diameters greater or equal to that of the filter pore. Accordingly, in some embodiments, the first solution and the second solution are each free of particles greater than 0.2 μM in diameter or free of particles greater than about 0.2 μM in diameter. In some embodiments, the first solution and the second solution are each substantially free of particles greater than 0.2 μM in diameter or substantially free of particles greater than about 0.2 μM in diameter.

In some embodiments, the peptide inhibitor comprises, consists essentially of, or consists of P28R (KKLDTFFVKLSLFTER; SEQ ID NO: 2). In some embodiments, the first solution comprises the peptide inhibitor at a concentration of at least 0.4 mg/ml, or at least about 0.4 mg/ml. In some embodiments, the first solution comprises a buffer having a buffer capacity of about 1.5 mM sodium acetate or less, or of 1.5 mM sodium acetate or less, for example, a buffer capacity of no more than that of 1.5 mM, 1.3 mM, 1.2 mM, 1 mM, 0.9 mM, 0.7 mM, or 0.5 mM sodium acetate. In some embodiments, the buffer comprises, consists essentially of, or consists of sodium acetate (and its conjugate acid). It is noted that buffer capacity can be compared to a reference (such as sodium acetate) under the same conditions (such as temperature and pressure). In some embodiments, buffer capacity is determined at standard temperature and pressure. It is shown herein that sodium (such as sodium acetate, sodium hydroxide, and/or sodium chloride) can case the peptide to form a gel, which can interfere with solubility and sterile filtration (see Example 11). Accordingly, it is contemplated that minimizing sodium content in the solution comprising peptide can facilitate solubility and sterile filtration. In some embodiments, the first solution does not comprise sodium acetate. In some embodiments, the first solution does not comprise a buffer. In some embodiments, the first solution does not comprise NaCl. In some embodiments, the first solution is substantially free of NaCl. In some embodiments, the first solution comprises, consists essentially of, or consists of less than or equal to 10 mM NaCl or less than or equal to about 10 mM NaCl but not zero. In some embodiments, the first solution is of the same volume, or about the same volume as the second solution.

In some embodiments, the pharmaceutical product comprises, consists essentially of, or consists of the first solution of Table 1A and the second solution of Table 1B. The first and second solution can be separate from each other. The first solution and second solution, when combined with each other, can produce a pharmaceutical formulation comprising, consisting essentially of, or consisting of the gel of Table 1C. As such, it will be appreciated that in accordance with some embodiments herein, a pharmaceutical formulation comprises, consists essentially of, or consists of the gel of Table 1C.

TABLE 1A
First Solution of Some Embodiments

Peptide inhibitor comprising, consisting essentially of, or consisting of the amino
acid sequence FFVKLS (SEQ ID NO: 1), such as P28R (KKLDTFFVKLSLFTER; (SEQ ID NO: 2)
at a concentration of at least 0.2 mg/ml or at least about 0.2 mg/ml, for example
at least 0.2, 0.4, 0.6, 0.8, 1, 1.2, 1.4, 1.6, 1.8, 2, 3, 4, or 5 mg/ml, or at
least about 0.2, 0.4, 0.6, 0.8, 1, 1.2, 1.4, 1.6, 1.8, 2, 3, 4, or 5 mg/ml.
pH less than 7 or about less than 7, for example, less than 7, less than 6, less
than 5.5, less than 5, less than 4.5, less than 4, less than 3.5, less than 3, or a
range between any tow of the listed values, for example, 3-4, 3-4.5, 3-5, 3-6,
3.5-4, 3.5-4.5, 3.5-5, 3.5-6, 3.5-6.5, 4-4.5, 4-5, 4-6, 4-6.5, 4.5-5, 4.5-6,
4.5-6.5, 4.5-7, 5-6, 5-6.5, 5-7, 5.5-6, 5.5-6.5, 5.5-7, 6-6.5, 6-7, or 6.5-7
Sub-isotonic osmolarity
Less than or equal to 10 mM NaCl or less than or equal to about 10 mM NaCl
Optionally, free or substantially free of particles greater than 0.2 μM in diameter
or free of particles greater than about 0.2 μM in diameter
Optional buffer having a buffer capacity that is equivalent to 1.5 mM sodium acetate
or less, or about 1.5 mM sodium acetate or less, but not zero

TABLE 1B
Second Solution of Some Embodiments

Tonicity Agent, such as sodium chloride
Base, such as sodium hydroxide
Above-isotonic osmolarity
Optionally, free or substantially free of particles greater than 0.2 μM
in diameter

TABLE 1C
Gel of Some Embodiments

Peptide inhibitor comprising, consisting essen-
tially of, or consisting of amino acid sequence
FFVKLS (SEQ ID NO: 1), such as P28R (KKLDTFFVKLSLF
TER; SEQ ID NO: 2), at a concentration of at least
0.2 mg/ml, for at least about 0.2 mg/ml, for
example at least 0.2, 0.4, 0.6, 0.8, 1, 1.2, 1.4,
1,6, 1.8, 2, 3, 4, or 5 mg/ml, or at least about
0.2, 0.4, 0.6, 0.8, 1, 1.2, 1.4, 1.6, 1.8, 2, 3,
4, or 5 mg/ml.
Tonicity Agent, such as sodium chloride or a non-
ionic tonicity agent
pH of 6.5-7.5, or about 6.5-7.5
Isotonic osmolarity or about isotonic osmolarity,
such as 280 -300 mOsmol/L, about 280 - 300 mOsmol/
L, 270 - 280 mOsmol/L, 270 - 290 mOsmol/L, 270 -
300 mOsmol/L, 270 - 310 mOsmol/L, 280 - 290
mOsmol/L, 280 - 300 mOsmol/L, 280 - 310 mOsmol/L,
290 - 300 mOsmol/L, 290 - 310 mOsmol/L, or 300 -
310 mOsmol/L.
Optionally, free or substantially free of parti-
cles greater than 0.2 μM in diameter

In some embodiments, the first solution comprises, consists essentially of, or consists of the solution of Table 1A, comprising a buffer having a buffer capacity equivalent to sodium acetate buffer at about 1.5 mM or less. The peptide inhibitor can comprise, consist essentially of, or consist of the amino acid sequence FFVKLS (SEQ ID NO: 1). The first solution can have a pH of less than about 7. The second solution can comprise, consist essentially of, or consist of the solution of Table 1B. The first and second solution, when combined with each other, can be configured to generate the gel of Table 1C, comprising the peptide inhibitor at a concentration of at least about 0.2 mg/ml, and having a pH of about 6.5-7.5, and an about isotonic osmolarity, such as about 270-310 mOsmol/L or about 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, comprising a buffer having a buffer capacity equivalent to sodium acetate buffer at 1.5 mM or less. The peptide inhibitor can comprise, consist essentially of, or consist of the amino acid sequence FFVKLS (SEQ ID NO: 1). The first solution can have a pH of less than 7. The second solution can comprise, consist essentially of, or consist of the solution of Table 1B. The first and second solution, when combined with each other, can form the gel of Table 1C, comprising the peptide inhibitor at a concentration of at least 0.2 mg/ml, and having a pH of 6.5-7.5, and an isotonic osmolarity, such as 270-310 mOsmol/L or 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free or substantially free of particles greater than 0.2 μM in diameter.

In some embodiments, the first solution comprises, consists essentially of, or consists of the solution of Table 1A, comprising a buffer having a buffer capacity equivalent to sodium acetate buffer at about 1.5 mM or less. The peptide inhibitor can comprise, consist essentially of, or consist of the amino acid sequence FFVKLS (SEQ ID NO: 1). The first solution can have a pH of less than about 5. The second solution can comprise, consist essentially of, or consist of the solution of Table 1B. The first and second solution, when combined with each other, can form the gel of Table 1C, comprising the peptide inhibitor at a concentration of at least about 0.2 mg/ml, and having a pH of about 6.5-7.5, and an about isotonic osmolarity, such as 270-310 mOsmol/L or 280-300 mOsmol/L. In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free or substantially free of particles greater than 0.2 μM in diameter.

In some embodiments, the first solution comprises, consists essentially of, or consists of the solution of Table 1A, comprising a buffer having a buffer capacity equivalent to sodium acetate buffer at 1.5 mM or less. The peptide inhibitor can comprise, consist essentially of, or consist of the amino acid sequence FFVKLS (SEQ ID NO: 1). The first solution can have a pH of less than 5. The second solution can comprise, consist essentially of, or consist of the solution of Table 1B. The first and second solution, when combined with each other, can form the gel of Table 1C, comprising the peptide inhibitor at a concentration of at least 0.2 mg/ml, and having a pH of 6.5-7.5, and an isotonic osmolarity, such as 270-310 mOsmol/L or 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, comprising a buffer having a buffer capacity equivalent to sodium acetate buffer at about 1.5 mM or less. The peptide inhibitor can comprise, consist essentially of, or consist of the amino acid sequence FFVKLS (SEQ ID NO: 1). The first solution can have a pH of about 3 to about 4.5. The second solution can comprise, consist essentially of, or consist of the solution of Table 1B. The first and second solution, when combined with each other, can form the gel of Table 1C, comprising the peptide inhibitor at a concentration of at least about 0.2 mg/ml, and having a pH of about 6.5-7.5, and an about isotonic osmolarity, such as about 270-310 mOsmol/L or about 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, comprising a buffer having a buffer capacity equivalent to sodium acetate buffer at 1.5 mM or less. The peptide inhibitor can comprise, consist essentially of, or consist of the amino acid sequence FFVKLS (SEQ ID NO: 1). The first solution can have a pH of 3 to 4.5. The second solution can comprise, consist essentially of, or consist of the solution of Table 1B. The first and second solution, when combined with each other, can form the gel of Table 1C, comprising the peptide inhibitor at a concentration of at least 0.2 mg/ml, and having a pH of 6.5-7.5, and an isotonic osmolarity, such as 270-310 mOsmol/L or 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, comprising sodium acetate buffer at about 1.5 mM or less. The peptide inhibitor can comprise, consist essentially of, or consist of the amino acid sequence FFVKLS (SEQ ID NO: 1). The first solution can have a pH of less than about 7. The second solution can comprise, consist essentially of, or consist of the solution of Table 1B. The first and second solution, when combined with each other, can form the gel of Table 1C, comprising the peptide inhibitor at a concentration of at least about 0.2 mg/ml, and having a pH of about 6.5-7.5, and an about isotonic osmolarity, such as 270-310 mOsmol/L or 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, comprising sodium acetate buffer at 1.5 mM or less. The peptide inhibitor can comprise, consist essentially of, or consist of the amino acid sequence FFVKLS (SEQ ID NO: 1). The first solution can have a pH of less than 7. The second solution can comprise, consist essentially of, or consist of the solution of Table 1B. The first and second solution, when combined with each other, can form the gel of Table 1C, comprising the peptide inhibitor at a concentration of at least 0.2 mg/ml, and having a pH of 6.5-7.5, and an isotonic osmolarity, such as 270-310 mOsmol/L or 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, comprising sodium acetate buffer at about 1.5 mM or less. The peptide inhibitor can comprise, consist essentially of, or consist of the amino acid sequence FFVKLS (SEQ ID NO: 1). The first solution can have a pH of less than about 5. The second solution can comprise, consist essentially of, or consist of the solution of Table 1B. The first and second solution, when combined with each other, can form the gel of Table 1C, comprising the peptide inhibitor at a concentration of at least about 0.2 mg/ml, and having a pH of about 6.5-7.5, and an about isotonic osmolarity, such as about 270-310 mOsmol/L or about 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, comprising sodium acetate buffer at 1.5 mM or less. The peptide inhibitor can comprise, consist essentially of, or consist of the amino acid sequence FFVKLS (SEQ ID NO: 1). The first solution can have a pH of less than 5. The second solution can comprise, consist essentially of, or consist of the solution of Table 1B. The first and second solution, when combined with each other, can form the gel of Table 1C, comprising the peptide inhibitor at a concentration of at least 0.2 mg/ml, and having a pH of 6.5-7.5, and an isotonic osmolarity, such as 270-310 mOsmol/L or 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, comprising sodium acetate buffer at about 1.5 mM or less. The peptide inhibitor can comprise, consist essentially of, or consist of the amino acid sequence FFVKLS (SEQ ID NO: 1). The first solution can have a pH of about 3.5 to 4.5. The second solution can comprise, consist essentially of, or consist of the solution of Table 1B. The first and second solution, when combined with each other, can form the gel of Table 1C, comprising the peptide inhibitor at a concentration of at least about 0.2 mg/ml, and having a pH of about 6.5-7.5, and an about isotonic osmolarity, such as 270-310 mOsmol/L or 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, and comprises sodium acetate buffer at 1.5 mM or less. The peptide inhibitor can comprise, consist essentially of, or consist of the amino acid sequence FFVKLS (SEQ ID NO: 1). The first solution can have a pH of 3.5 to 4.5. The second solution can comprise, consist essentially of, or consist of the solution of Table 1B. The first and second solution, when combined with each other, can form the gel of Table 1C, comprising the peptide inhibitor at a concentration of at least 0.2 mg/ml, and having a pH of 6.5-7.5, and an isotonic osmolarity, such as 270-310 mOsmol/L or 280-300 mOsmol/L In some embodiments, the peptide inhibitor comprises, consists essentially of, or consists of the amino acid sequence of P28R (KKLDTFFVKLSLFTER; SEQ ID NO: 2). In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, and comprises a buffer having a buffer capacity equivalent to sodium acetate buffer at about 1.5 mM or less. The peptide inhibitor can comprise, consist essentially of, or consist of the amino acid sequence of P28R (KKLDTFFVKLSLFTER; SEQ ID NO: 2). The first solution can have a pH of less than about 5, for example less than about 5, 4.5, 4, 3.5, or 3, including ranges between any two of the listed values, for example, about 3-5, about 3.5-5, about 4-5, about 4.5-5, about 3-4.5, about 3.5-4.5, about 4-4.5, about 3-4, or about 3.5-4. The second solution can comprise, consist essentially of, or consist of the solution of Table 1B. The first and second solution, when combined with each other, can form the gel of Table 1C, comprising the peptide inhibitor at a concentration of at least 0.2 mg/ml (such as at least 0.4 mg/ml), and having a pH of about 6.5-7.5, and an about isotonic osmolarity, such as 270-310 mOsmol/L or 280-300 mOsmol/L. In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, and comprises a buffer having a buffer capacity equivalent to sodium acetate buffer at 1.5 mM or less. The peptide inhibitor can comprise, consist essentially of, or consist of the amino acid sequence of P28R (KKLDTFFVKLSLFTER; SEQ ID NO: 2). The first solution can have a pH of less than about 5, for example less than 5, 4.5, 4, 3.5, or 3, including ranges between any two of the listed values, for example, 3-5, 3.5-5, 4-5, 4.5-5, 3-4.5, 3.5-4.5, 4-4.5, 3-4, or 3.5-4. The second solution can comprise, consist essentially of, or consist of the solution of Table 1B. The first and second solution, when combined with each other, can form the gel of Table 1C, comprising the peptide inhibitor at a concentration of at least 0.2 mg/ml (such as at least 0.4 mg/ml), and having a pH of 6.5-7.5, and an isotonic osmolarity, such as 270-310 mOsmol/L or 280-300 mOsmol/L In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, and comprises sodium acetate buffer at about 1.5 mM or less. The peptide inhibitor can comprise, consist essentially of, or consist of the amino acid sequence of P28R (KKLDTFFVKLSLFTER; SEQ ID NO: 2). The first solution can have a pH of less than about 5, for example less than about 5, 4.5, 4, 3.5, or 3, including ranges between any two of the listed values, for example, about 3-5, about 3.5-5, about 4-5, about 4.5-5, about 3-4.5, about 3.5-4.5, about 4-4.5, about 3-4, or about 3.5-4. The second solution can comprise, consist essentially of, or consist of the solution of Table 1B. The first and second solution, when combined with each other, can form the gel of Table 1C, comprising the peptide inhibitor at a concentration of at least about 0.2 mg/ml (such as at least about 0.4 mg/ml), and having a pH of about 6.5-7.5, and an about isotonic osmolarity, such as 270-310 mOsmol/L or 280-300 mOsmol/L. In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, and comprises sodium acetate buffer at 1-1.5 mM. The peptide inhibitor can comprise, consist essentially of, or consist of the amino acid sequence of P28R (KKLDTFFVKLSLFTER; SEQ ID NO: 2). The first solution can have a pH of less than about 5, for example less than 5, 4.5, 4, 3.5, or 3, including ranges between any two of the listed values, for example, 3-5, 3.5-5, 4-5, 4.5-5, 3-4.5, 3.5-4.5, 4-4.5, 3-4, or 3.5-4. The second solution can comprise, consist essentially of, or consist of the solution of Table 1B. The first and second solution, when combined with each other, can form the gel of Table 1C, comprising the peptide inhibitor at a concentration of at least 0.2 mg/ml (such as at least 0.4 mg/ml), and having a pH of 6.5-7.5, and an isotonic osmolarity, such as 270-310 mOsmol/L or 280-300 mOsmol/L. In some embodiments, the first solution and the second solution are free of particles greater than 0.2 μM in diameter or free of particles greater than about 0.2 μM in diameter.

In some embodiments, the first solution comprises, consists essentially of, or consists of the solution of Table 1A, and comprises a buffer having a buffer capacity equivalent to sodium acetate buffer at about 1.5 mM. The peptide inhibitor can have the amino acid sequence of P28R (KKLDTFFVKLSLFTER; SEQ ID NO: 2). The first solution can have a pH of 3-4.5. The second solution can comprise, consist essentially of, or consist of the solution of Table 1B. The first and second solution, when combined with each other, can form the gel of Table 1C, comprising the peptide inhibitor at a concentration of at least 0.2 mg/ml (such as at least about 0.4 mg/ml), and having a pH of 6.5-7.5, and an isotonic osmolarity, such as 270-310 mOsmol/L or 280-300 mOsmol/L. In some embodiments, the first solution can comprise no more than about 10 mM NaCl. The tonicity agent of the second solution can comprise NaCl. Without being limited by theory, it is contemplated that by providing NaCl in the second solution (for tonicity, and/or other purposes), sodium can be minimized in the first solution, thus minimizing gel formation by the peptide inhibitor in the first solution. In some embodiments, the tonicity agent of the second solution is NaCl, and the gel of Table 1C comprises the NaCl at 100 mM-120 Mm NaCl, or about 100 mM-120 mM NaCl. In some embodiments, the first solution is free or substantially free of gel. In some embodiments, the peptide inhibitor in the first solution is not in the beta sheet conformation, or is substantially free of peptide inhibitor in the beta sheet conformation. In some embodiments, the first solution and the second solution are free of particles greater than 0.2 μM in diameter or free of particles greater than about 0.2 μM in diameter.

In some embodiments, for any pharmaceutical product described herein, the first solution comprises less than or equal to about 10 mM NaCl, for example less than or equal to about 10 mM NaCl but not zero, for example, than or equal to about 10, 9, 8, 7, 56, 5, 4, 3, 2, 1, or 0.1 mM NaCl, including ranges between any two of the listed values, for example about 0.1-10 mM NaCl, about 0.1-7 mM NaCl, about 1-7 mM NaCl, about 1-10 mM NaCl, about 5-7 mM NaCl, or about 5-10 mM NaCl. In some embodiments, for any pharmaceutical product described herein, the first solution comprises less than or equal to 10 mM NaCl, for example, less than or equal to 10, 9, 8, 7, 56, 5, 4, 3, 2, 1, or 0.1 mM NaCl, including ranges between any two of the listed values, for example 0.1-10 mM NaCl, 0.1-7 mM NaCl, 1-7 mM NaCl, 1-10 mM NaCl, 5-7 mM NaCl, or 5-10 mM NaCl. In some embodiments, for any pharmaceutical product described herein, the first solution is substantially free of NaCl. In some embodiments, for any pharmaceutical product described herein, the first solution does not comprise NaCl.

In some embodiments, for any pharmaceutical product described herein, the first solution comprises a buffer having a buffer capacity that is equivalent to 1.5 mM or less, or about 1.5 mM sodium acetate or less, but not zero, for example, less than or equal to 1.5 mM, 1.3 mM, 1.1 mM 1 mM, 0.7 mM, or 0.5 mM sodium acetate (but not zero), including ranges between any two of the listed values. In some embodiments, for any pharmaceutical product described herein, the first solution does not comprise a buffer. For example, in some embodiments, the first solution of Table 1A does not comprise a buffer or is substantially free of buffer. In some embodiments, for any pharmaceutical product described herein, the first solution is substantially free of buffer. It is noted that the pharmaceutical product that is free of substantially free of buffer may have buffer capacity attributable to the peptide inhibitor itself, but is free or substantially free of a small molecule buffer such as sodium acetate.

In some embodiments, for any pharmaceutical product described herein, the first solution comprises sodium acetate at a concentration of 1.5 mM or less or about 1.5 mM or less, but not zero, for example, less than or equal to 1.5 mM, 1.3 mM, 1.1 mM 1 mM, 0.7 mM, or 0.5 mM sodium acetate (but not zero), including ranges between any two of the listed values. In some embodiments, for any pharmaceutical product described herein, the first solution does not comprise sodium acetate. In some embodiments, for any pharmaceutical product described herein, the first solution is substantially free of sodium acetate.

In some embodiments, for any pharmaceutical product or formulation described herein, the tonicity agent comprises, consists essentially of, or consists of NaCl. In some embodiments, the second solution is configured for the gel to comprise 100 mM-120 mM NaCl or about 100 mM-120 mM NaCl. In some embodiments, the second solution is configured for the gel to comprise 90 mM-130 mM NaCl, about 90 mM-130 mM NaCl, 110 mM-120 mM NaCl, about 110 mM-120 mM NaCl, 100 mM-110 mM NaCl, or about 100 mM-110 mM NaCl. In some embodiments, for any pharmaceutical product described herein, the first solution does not comprise NaCl. In some embodiments, the pharmaceutical product or pharmaceutical formulation as described herein is substantially free of NaCl. In some embodiments, the pharmaceutical product or pharmaceutical formulation does not comprise NaCl. Accordingly, the tonicity agent of the second solution can be a tonicity agent other than NaCl, for example a non-ionic toxicity agent.

In some embodiments, for any pharmaceutical product described herein, the first solution is free of gel, or substantially free of gel. In some embodiments, less than 5%, 4%, 3%, 2%, 1%, 0.5%, or 0.1% of the peptide inhibitor of the first solution is in a gel. In some embodiments, for any pharmaceutical product described herein, the peptide inhibitor is dissolved in the first solution. In some embodiments, for any pharmaceutical product described herein, the peptide inhibitor is dissolved in the first solution, and is substantially not, or is not in a beta-sheet conformation. In some embodiments, for any pharmaceutical product described herein, the peptide inhibitor is dissolved in the first solution, and less than 5%, 4%, 3%, 2%, 1%, 0.5%, or 0.1% of the peptide inhibitor of the first solution is in a beta sheet conformation.

In some embodiments, for any pharmaceutical product described herein, the first solution is configured for the gel of the pharmaceutical formulation to comprise the peptide inhibitor at a concentration of at least 0.4 mg/ml or at least about 0.4 mg/ml. In some embodiments, for any pharmaceutical product described herein, the first solution is configured for the gel to comprise the peptide inhibitor at a concentration of at least 0.4 mg/ml, such at least 0.4, 0.5, 0.6, 0.8, or 1 mg/ml, including ranges between any two of the listed values.

In some embodiments, for any pharmaceutical product or pharmaceutical formulation described herein, the peptide inhibitor comprises no more than 30 amino acid residues, for example, no more than 30, 29, 28, 27, 25, 26, 25, 24, 23, 22, 21, 20, 19, 18, 17, 16, 15, 14, 13, 12, 11, or 10 amino acid residues, including ranges between any two of the listed values.

In some embodiments, for any pharmaceutical product or pharmaceutical formulation described herein, the peptide inhibitor comprises the amino acid sequence KKLDTFFVKLSLFTER (SEQ ID NO: 2). In some embodiments, for any pharmaceutical product described herein, the peptide inhibitor consists of the amino acid sequence KKLDTFFVKLSLFTER (SEQ ID NO: 2).

In some embodiments, the first solution is capable of maintaining at least 95% or at least about 95% of the peptide inhibitor dissolved in said first solution at 5° C. for at least 12-25 months or at least about 12-25 months, for example when at least 95%, 96%, 97%, 98%, or 99% of the peptide inhibitor dissolved. In some embodiments, the first solution is capable of maintaining at least 95% or at least about 95% of the peptide inhibitor dissolved in said first solution at 5° C. for at least 12-19 months or at least about 12-19 months, for example at least within 95%, 96%, 97%, 98%, or 99% of the peptide inhibitor dissolved.

In some embodiments, the first solution and the second solution are configured for the gel to comprise at least 0.4 mg/ml or at least about 0.4 mg/ml of the peptide inhibitor consisting of the amino acid sequence KKLDTFFVKLSLFTER (SEQ ID NO: 2). The first solution and the second solution can be further configured for the gel to comprise 30-40 mM or at least about 30-40 mM acetic acid; 1.2-1.6 mM or at least about 1.2-1.6 mM sodium acetate; less than or equal to 30 mM or less than or equal to about 30 mM sodium hydroxide; and 100-120 mM or about 100-120 mM sodium chloride. The gel can have an osmolarity of 280-300 mOSmoL/L or about 280-300 mOSmol/L.

### Pharmaceutical Formulations

### Gel Formulations

In some embodiments, pharmaceutical formulations are described. The pharmaceutical formulation can comprise, consist essentially of, or consist of a gel, for example a gel of Table 1C. The gel can comprise at least or at least about 0.4 mg/ml of an isolated peptide comprising, consisting essentially of, or consisting of the amino acid sequence FFVKLS (SEQ ID NO: 1). The gel can comprise a buffer system comprising acetic acid and sodium acetate, the buffer system comprising less than or equal to 1.6 mM or less than or equal to about 1.6 mM sodium acetate. The gel can comprise a tonicity agent. The gel can be isotonic and/or can have a pH of 4.5-7.5 or about 4.5-7.5. In some embodiments, the pharmaceutical formulation is for medical use. In some embodiments, the pharmaceutical formulation is for use in ameliorating, inhibiting, reducing the symptoms of, or treating a cancer as described herein.

As noted herein, while gels can interfere with sterile filtration, precursor solutions can be sterile filtered, for example via filters having a pore size of 0.2 μM or less, for example, 0.2 μM or less, or 0.1 μM or less. As such, in some embodiments, the gel is substantially free of particles greater than 0.2 μM in diameter or greater than about 0.2 μM in diameter. In some embodiments, the gel is free of particles greater than 0.2 μM or greater than about 0.2 μM in diameter. In some embodiments, the gel is free of particles greater than 0.1 μM or greater than about 0.1 μM in diameter.

In some embodiments, for any pharmaceutical formulation comprising a gel as described herein, the tonicity agent of the gel comprises, consists essentially of, or consists of NaCl. The gel can comprise the tonicity agent (NaCl) at a concentration of 100-120 mM or about 100-120 mM. In some embodiments, the gel comprises the NaCl at a concentration of no more than 50, 60, 70, 80, 90, 100, 105, 110, 115, 120, 130, 140, or 150 mM, including ranges between any two of the listed values, for example, 50-150 mM. In some embodiments, the gel comprises the NaCl at a concentration of 105-115 mM or about 105-115 mM. In some embodiments, the gel comprises the NaCl at a concentration of about 110 mM.

In some embodiments, for any pharmaceutical formulation comprising a gel as described herein, the buffer system has a buffer capacity of no more than that of 20 mM sodium acetate buffer, for example, no more than 20, 15, 10, 5, 4, 3, 2, or 1 mM sodium acetate, including ranges between any two of the listed values, for example, 1-2 mM, 1-5 mM, 1-10 mM, 1-20 mM, 2-5 mM, 2-10 mM, 2-20 mM, 5-10 mM, or 5-20 mM, In some embodiments, the buffer system comprises 30-40 mM acetic acid and 1.2-1.6 mM sodium acetate, or about 30-40 mM acetic acid and about 1.2-1.6 mM sodium acetate. In some embodiments, the buffer system does not comprise sodium acetate. In some embodiments, the buffer system is substantially free of sodium acetate. In some embodiments, any pharmaceutical formulation as described herein is substantially free of buffer. In some embodiments, any pharmaceutical formulation as described herein does not comprise buffer.

In some embodiments, any pharmaceutical formulation described herein comprises sodium hydroxide. The sodium hydroxide can be at a concentration of less than or equal to 30 mM or less than or equal to about 30 mM but not zero, for example, less than or equal to 30 mM, 25 mM, 20 mM, 15 mM, 10 mM, or 1 mM, including ranges between any two of the listed values.

In some embodiments, for any pharmaceutical formulation comprising a gel as described herein, the gel has a tonic osmolarity. In some embodiments, the gel has an osmolarity of 270-310 mOSmol/L, about 270-310 mOSmol/L, 280-300 mOSmol/L, about 280-300 mOSmol/L, 285-295 mOSmol/L, about 285-295 mOSmol/L, 289-291 mOSmol/L, or about 289-291 mOSmol/L.

In some embodiments, for any pharmaceutical formulation comprising a gel as described herein, the peptide inhibitor comprises no more than 30 amino acid residues, for example, no more than 30, 29, 28, 27, 25, 26, 25, 24, 23, 22, 21, 20, 19, 18, 17, 16, 15, 14, 13, 12, 11, or 10 amino acid residues, including ranges between any two of the listed values.

In some embodiments, for any pharmaceutical formulation comprising a gel as described herein, the peptide inhibitor comprises the amino acid sequence KKLDTFFVKLSLFTER (SEQ ID NO: 2). In some embodiments, for any pharmaceutical product described herein, the peptide inhibitor consists of the amino acid sequence KKLDTFFVKLSLFTER (SEQ ID NO: 2).

### Single-Solution Pharmaceutical Formulations

In some embodiments, the pharmaceutical formulation comprises, consists essentially of, or consists of a liquid. It has been shown herein that peptide inhibitors can be formulated at relatively high concentrations, for example, up to about 10 mg/ml (See Example 14). Without being limited by theory, it is contemplated that while sodium and/or pH's above 4.5 can cause peptide inhibitors to precipitate (See Examples 10-11), in higher concentration formulations, the peptide inhibitor itself can act as a weak buffer (for example, due to interaction between acid side chains on the peptide inhibitor), thus permitting low levels of sodium. The isolated peptide of the pharmaceutical formulation can comprise the amino acid sequence FFVKLS (SEQ ID NO: 1). The isolated peptide can be dissolved in the pharmaceutical formulation at a concentration of 0.2-20 mg/ml, or at about 0.2-20 mg/ml. The pharmaceutical formulation can further comprise a non-ionic tonicity agent. The pharmaceutical formulation can be isotonic. In some embodiments, the pharmaceutical formulation is isotonic, and has a pH of 5.0-5.5 or about 5.0-5.5. In some embodiments, the pharmaceutical formulation is for medical use. In some embodiments, the pharmaceutical formulation is for use in ameliorating, inhibiting, reducing the symptoms of, or treating a cancer as described herein.

In some embodiments, for any pharmaceutical formulation described herein, the pharmaceutical composition is free or substantially free of particles having a diameter greater than or greater than about 0.2 μM, for example particles having diameters greater than 0.1 μM.

In some embodiments, for any pharmaceutical formulation described herein, the pharmaceutical formulation further comprises a weak acid. The peptide and the weak acid can comprise a buffer system that maintains the pharmaceutical formulation at a pH of 4.5-5, for example about 4.5-5, 4.5-5.5, 4.5-6, 4.5-6.5, 4.5-7, 5-5.5, 5-6, 5-6.5, 5-7, 5.5-6, 5.5-6.5, 5.5-7, 6-6.5, or 6-7,

In some embodiments, for any pharmaceutical formulation described herein, the weak acid is acetic acid, which is present at a concentration of no more than 0.02 M or about 0.02M, for example, no more than 0.02 M, about 0.02M, 0.01M, about 0.01M, 0.005M, or about 0.005M.

In some embodiments, for any pharmaceutical formulation described herein, the non-ionic tonicity agent is glucose. In some embodiments, the non-ionic tonicity agent is glucose, and is present at a concentration effective for the pharmaceutical formulation to be tonic. In some embodiments, the non-ionic tonicity agent is glucose, and is present at a concentration of 0.1-0.5 M, about 0.1-0.5 M, 0.2-0.4 M, about 0.2-0.4M, 0.2-0.3M, about 0.2-0.3M, 0.3-0.4M, or about 0.3-0.4M.

In some embodiments, for any pharmaceutical formulation comprising, consisting essentially of, or consisting of a liquid as described herein the isolated peptide is at a concentration of 0.2-5 mg/ml, about 0.2-5 mg/ml, 0.2-10 mg/ml, about 0.2-10 mg/ml, 0.2-20 mg/ml, about 0.2-20 mg/ml, 1-5 mg/ml, about 1-5 mg/ml, 1-10 mg/ml, about 1-10 mg/ml, 1-20 mg/ml, about 1-20 mg/ml, 5-10 mg/ml, about 5-10 mg/ml, 5-20 mg/ml, or about 5-20 mg/ml. In some embodiments, the isolated peptide is at a concentration of 10 mg/ml or less, for example a concentration of no more than 10 mg/ml, about 10 mg/ml, 5 mg/ml, about 5 mg/ml, 1 mg/ml, or about 1 mg/ml.

In some embodiments, for any pharmaceutical formulation as described herein, the peptide inhibitor comprises no more than 30 amino acid residues, for example, no more than 30, 29, 28, 27, 25, 26, 25, 24, 23, 22, 21, 20, 19, 18, 17, 16, 15, 14, 13, 12, 11, or 10 amino acid residues, including ranges between any two of the listed values.

In some embodiments, for any pharmaceutical formulation comprising, consisting essentially of, or consisting of a liquid as described herein, the peptide inhibitor comprises the amino acid sequence KKLDTFFVKLSLFTER (SEQ ID NO: 2). In some embodiments, for any pharmaceutical formulation comprising, consisting essentially of, or consisting of an liquid as described herein, the peptide inhibitor consists of the amino acid sequence KKLDTFFVKLSLFTER (SEQ ID NO: 2).

In some embodiments, any pharmaceutical formulation comprising, consisting essentially of, or consisting of a liquid as described herein further comprises 0.01 M or about 0.01M acetic acid. The isolated peptide consists of the amino acid sequence KKLDTFFVKLSLFTER (SEQ ID NO: 2). The peptide inhibitor can be present at a concentration of 1-10 mg/ml or about 1-10 mg/ml. The non-ionic tonicity agent can be glucose, which can be present at a concentration of 0.2-0.4M or about 0.2-0.4 M. The pharmaceutical formulation can be isotonic as described herein, for example having an osmolarity of about 280-300 mOsm/L or 280-300 mOsm/L.

### Methods of Preparing a Pharmaceutical Formulation

Some embodiments include a method of preparing a pharmaceutical formulation from the pharmaceutical product as described herein. The method can comprise combining a first solution as described herein (for example a first solution of Table 1A) with a second solution as described herein (for example, a second solution of Table 1B) to form a gel (such as a gel of Table 1C). The pharmaceutical formulation can comprise, consist essentially of, or consist of the gel.

### Methods of Manufacturing Pharmaceutical Products

In some embodiments, a method of manufacturing a pharmaceutical product is described. The method can comprise sterile filtering a solution comprising a peptide inhibitor comprising the amino acid sequence FFVKLS (SEQ ID NO: 1) as described herein. The peptide inhibitor can be dissolved in the precursor solution at a concentration of at least or at least about 0.2 mg/ml, 0.3 mg/ml, 0.4 mg/ml, 0.5 mg/ml, 0.6 mg/ml, 0.7 ml/ml, 0.8 mg/ml, 0.9 mg/ml, or 1 mg/ml, including ranges between any two of the listed values. It will be appreciate that high pH and/or high sodium can cause peptide inhibitors as described herein to form a gel (See Examples 10-11). Accordingly, it will be appreciated that in some embodiments herein, a peptide inhibitor is dissolved in a precursor solution (such as a solution of Table 1A) selected to avoid gel formation prior to sterile filtration, for example a solution comprising an acidic pH such as less than about 5, 4.5, 4, 3.5, 3, 2.5, or 2 or a range between any two of the listed values, for example 2-3, 2-3.5, 2-4, 2-4.5, 2-5, 3-3.5, 3-4, 3-4.5, 3-5, 3.5-4, 3.5-4.5, 3.5-5, 4-4.5, or 4-5; and less than or equal to 10 mM NaCl or less than or equal to about 10 mM NaCl; and less than or equal to 1.5 mM NaCl or less than or equal to about 1.5 mM NaCl or less but not zero. In some embodiments, the pH, NaCl, and sodium acetate content of the solution are such that the peptide inhibitor remains dissolved in the precursor solution, and the precursor solution is free or substantially free of gel.

In some embodiments, the precursor solution comprising the peptide inhibitor is sterile-filtered with a filter having a pore size of about 0.2 μM or 0.2 μM. It will be appreciated at a pH greater than 4.5, or in the presence of levels of sodium chloride (See Examples 10-11), the peptide inhibitor can form a gel, which interferes with sterile filtration. It will further be appreciated that a sterile-filtered precursor solution will be free or substantially free of particles having diameters at least about the size of the pore size of a filter. For example, it will be appreciated that a precursor solution comprising the peptide inhibitor that is sterile-filtered with a filter of pore size of 0.2 μM will be free or substantially free of particles greater than 0.2 μM in diameter. In some embodiments, the precursor solution comprising the peptide inhibitor is sterile-filtered with a filter of pore size of no more than 1 μM, about 1 μM, 0.5 μM, about 0.5 μM, 0.4 μM, about 0.4 μM, 0.3 μM, about 0.3 μM, 0.2 μM, about 0.2 μM, 0.1 μM, or about 0.1 μM, including ranges between any two of the listed values.

It has been shown that at elevated pH, peptide inhibitors can form a gel (Example 11). In the method of some embodiments, the precursor solution has an acidic pH. The pH can be sufficient to inhibit formation of a gel by the peptide inhibitor. In the method of some embodiments, the precursor solution has a pH of less than about 5, 4.5, 4, 3.5, 3, 2.5, or 2, including ranges between any two of the listed values, for example, 2-3, 2-3.5, 2-4, 2-4.5, 2-5, 3-3.5, 3-4, 3-4.5, 3-5, 3.5-4, 3.5-4.5, 3.5-5, 4-4.5, or 4-5.

In the method of some embodiments, the peptide inhibitor comprises no more than 30 amino acid residues, for example, no more than 30, 29, 28, 27, 25, 26, 25, 24, 23, 22, 21, 20, 19, 18, 17, 16, 15, 14, 13, 12, 11, or 10 amino acid residues, including ranges between any two of the listed values. In the method of some embodiments, the peptide inhibitor comprises the amino acid sequence KKLDTFFVKLSLFTER (SEQ ID NO: 2). In the method of some embodiments, the peptide inhibitor consists of the amino acid sequence KKLDTFFVKLSLFTER (SEQ ID NO: 2).

### Methods of Ameliorating, Inhibiting, Reducing the Symptoms of, or Treating a Cancer

In some embodiments, a method of ameliorating, inhibiting, reducing the symptoms of, or treating a cancer in a patient in need thereof is described. The method can comprise administering an effective amount of the pharmaceutical formulation as described herein to the patient. It will be appreciated that whenever a method of ameliorating, inhibiting, reducing the symptoms of, or treating a cancer is described herein (and the method comprises a pharmaceutical formulation), the corresponding pharmaceutical formulation for use in ameliorating, inhibiting, reducing the symptoms of, or treating the cancer is also expressly contemplated. The “patient” in need of ameliorating, inhibiting, reducing the symptoms of, or treating a cancer may also be referred to herein as a “subject.”

In methods of some embodiments, the effective amount of the pharmaceutical formulation refers to an amount of pharmaceutical formulation comprising a sufficient amount of peptide inhibitor to ameliorate, inhibit, reduce the symptoms of, or treat the cancer in the patient. In methods of some embodiments, the effective amount of the pharmaceutical formulation comprises an amount of peptide inhibitor sufficient to cause immune cell infiltration of a tumor. In methods of some embodiments, the effective amount of the pharmaceutical formulation comprises an amount of peptide inhibitor sufficient to cause cell death in a tumor. In methods of some embodiments, the effective amount of pharmaceutical formulation comprises up to 5 mg/kg of the peptide inhibitor, for example, up to 0.01, 0.02, 0.05, 0.09, 0.1, 0.2, 0.3, 0.4, 0.5, 0.6, 0.7, 0.08, 0.9, 1, 2, 3, 4, or 5 mg/kg, including ranges between any two of the listed values. In methods of some embodiments, the effective amount of pharmaceutical formulation comprises up to about 5 mg/kg or 1 mg/kg of the peptide inhibitor. In methods of some embodiments, the effective amount comprises up to 2000 μg or up to about 2000 μg of peptide inhibitor, for example, up to 1, 2, 3, 4, 5, 6, 7, 8, 9, 10, 20, 30, 40, 50, 60, 70, 80, 90, 100, 150, 200, 250, 300, 350, 400, 450, 500, 550, 600, 650, 700, 750, 800, 850, 900, 1000, 1100, 1200, 1300, 1400, 1500, 1600, 1700, 1800, 1900, or 2000 μg, including ranges between any two of the listed values, for example, 1-50 μg, 1-80 μg, 1-100 μg, 1-500 μg, 1-800 μg, 1-1000 μg, 1-2000 μg, 8-50 μg, 8-80 μg, 8- 100 μg, 8-500 μg, 8-800 μg, 8-1000 μg, 8-2000 μg, 10-50 μg, 10-80 μg, 10-100 μg, 10-500 μg, 10-800 μg, 10-1000 μg, 10-2000 μg, 50-80 μg, 50-100 μg, 50-500 μg, 50-800 μg, 50-1000 μg, 50-2000 μg, 100-500 μg, 100-800 μg, 100-1000 μg, 100-2000 μg, 500-800 μg, 500-1000 μg, or 500-2000 μg of the peptide inhibitor. In methods of some embodiments, the effective amount of the pharmaceutical formulation comprises 8-800 μg or about 8-800 μg of the peptide inhibitor. In methods of some embodiments, the effective amount of the pharmaceutical formulation comprises 60-100 μg or about 60-100 μg of the peptide inhibitor.

Any number of suitable routes of administration can be used in methods of some embodiments herein. In some embodiments, the pharmaceutical formulation is administered intratumorally, subcutaneously, lymphatically, and/or to an interstitial fluid of the patient.

In the method of some embodiments, administration of the pharmaceutical formulation is repeated. The administration can be performed at least 2, 3, 4, 5, 6, 7, 8, 9, 10, 15, 20, 30, 40, or 50 times, including ranges between any two of the listed values. In some embodiments, administration of the pharmaceutical formulation is repeated over a period of time, for example repeated at least every 1, 2, 3, 4, 5, 6, 7, 8, 9, or 10 weeks.

In the method of some embodiments, the cancer comprises, consists essentially of, or consists of a tumor. In the method of some embodiments, the cancer is selected from the group consisting of; head and neck cancer, breast cancer, renal cancer, colorectal cancer, skin cancer, ovarian cancer, prostate cancer, pancreatic cancer, lung cancer, malignant melanoma, small cell lung cancer, non-small lung cancer (adenocarcinoma), squamous cell carcinoma, bladder cancer, osteosarcoma, bronchial cancer, and/or hematopoietic cell cancer. In some embodiments, the patient has two or more of the listed cancers.

It will be appreciated that the peptide inhibitors of some embodiments can alleviate immunosuppression associated with P3028 structures (See Examples 1-3). Accordingly, in the method of some embodiments, the patient is selected as having P3028 structures, and/or immunosuppression of P3028 structures. It is contemplated that this selection can identify the patient as a candidate for treatment with a pharmaceutical composition comprising a peptide inhibitor as described herein (See Example 7). In some embodiments, the method comprises detecting the presence and/or level of peptide P3028 (SEQ ID NO: 3) or a P3028 structure such as damaged or denatured albumin in a sample of a patient, such as hematopoietic tissue, a body fluid, a blood sample, a tumor biopsy, or a biopsy of tissue surrounding the tumor. In some embodiments, the method comprises detecting the presence and/or level of a P3028 structure such as damaged or denatured albumin in a sample of a patient, such as hematopoietic tissue, a body fluid, a blood sample, a tumor biopsy, or a biopsy of tissue surrounding the tumor. The patient can be selected for treatment with the pharmaceutical composition comprising a peptide inhibitor if the patient comprises a presence and or level of the peptide P3028 (SEQ ID NO: 3) or a P3028 structure greater than that of a healthy (non-immunosuppressed) control. In some embodiments, the method comprises receiving the results of detection of a presence and/or level of peptide P3028 (SEQ ID NO: 3) or a P3028 structure such as damaged or denatured albumin in a sample of a patient, such as a sample comprising hematopoietic tissue, a body fluid, a blood sample, a tumor biopsy, or a biopsy of tissue surrounding the tumor. In some embodiments, the method comprises receiving the results of detection of a presence and/or level a P3028 structure such as damaged or denatured albumin in a sample of a patient, such as a sample comprising hematopoietic tissue, a body fluid, a blood sample, a tumor biopsy, or a biopsy of tissue surrounding the tumor. The patient can be selected for treatment with the pharmaceutical composition comprising a peptide inhibitor if the patient comprises a presence and or level of the peptide P3028 (SEQ ID NO: 3) or a P3028 structure greater than that of a healthy (non-immunosuppressed) control. In some embodiments, the sample comprises hematopoietic tissue, a blood sample, a blood sample, a tumor biopsy, or a biopsy of tissue surrounding the tumor.

It has further been observed that the presence of immune cells in a tumor can indicate alleviation of immunosuppression, and an immune response to a tumor (See, e.g., Examples 4-5). Accordingly, detection of a presence and/or level of immune cells in a tumor of a patient in methods of some embodiments can indicate that the pharmaceutical formulation is effectively ameliorating, inhibiting, reducing the symptoms of, or treating a cancer in the patient. In some embodiments, the method comprises detecting a presence of immune cells in a sample of the patient, such as a tumor biopsy. In some embodiments, the method comprises receiving results of detection of a presence of immune cells in a sample of the patient, such as a tumor biopsy. The sample of the patient, such as the tumor biopsy, can have been collected from the patient at least 5 days after the administration of the pharmaceutical composition as described herein, for example at least 5, 6, 7, 8, 9, 10, 15, or 20 days after.

It has been observed that some tumor microenvironments comprise a substantial presence of P3028 structures, and absence of immune cells (e.g., “immune dessert” and “immune excluded” cancer; See Example 7). Moreover, it has been observed that P3028 levels in tumors inversely correlate with T cell infiltration. It is contemplated that this sort of “immune dessert” and “immune excluded” profile can be indicative of a patient amenable to treatment using a pharmaceutical composition comprising a peptide inhibitor as described herein. In some embodiments, the method comprises selecting the patient as comprising a presence or level of a P3028 structure (such as peptide P3028, denatured albumin, and/or damaged albumin) and/or a presence or absence immune cells in a sample of the patient. The sample can comprise hematopoietic tissue, a body fluid, a blood sample, or a tumor biopsy. Such a patient can be selected to receive an effective amount of the pharmaceutical formulation. In some embodiments, the method comprises selecting the patient for receiving an effective amount of the pharmaceutical formulation if immune cells are absent from a tumor in a sample of the patient. The sample can comprise a hematopoietic tissue, a body fluid, a blood sample, and/or a tumor biopsy. In some embodiments, the method comprises selecting the patient for receiving an effective amount of the pharmaceutical formulation if immune cells are absent from a sample of the patient that comprises a tumor and P3028 structures are in a sample of the patient. The sample can comprise a hematopoietic tissue, a body fluid, a blood sample, and/or a tumor biopsy. In some embodiments, the method comprises selecting the patient for receiving an effective amount of the pharmaceutical formulation if immune cells such as T cells are absent from a sample of the patient that comprises a tumor. The sample can comprise a hematopoietic tissue, a body fluid, a blood sample, and/or a tumor biopsy. In some embodiments, the method comprises selecting the patient for receiving an effective amount of the pharmaceutical formulation if P3028 structures are in a sample of the patient. The sample can comprise a hematopoietic tissue, a body fluid, a blood sample, and/or a tumor biopsy. In some embodiments, the method comprises selecting the patient for receiving an effective amount of the pharmaceutical formulation if the patient has a tumor that is substantially free of T cell infiltrates, a tumor that comprises a majority of T-cell infiltrates in the stroma, or a tumor that is inflamed and infiltrated by inactive T cells. In some embodiments the method further comprises detecting an inflammatory response to the tumor after administration of the pharmaceutical formulation, such as effector cell and/or suppressor cell infiltration of the tumor. In some embodiments, the sample comprises a hematopoietic tissue, a blood sample, and/or a tumor biopsy.

Death of tumor cells can also be indicative of alleviation of immunosuppression and inhibition of tumors in response to a pharmaceutical composition as described herein (See, e.g., Example 6). Accordingly, in some embodiments, the method further comprises detecting death of tumor cells, such as apoptosis or necrosis, after administration of the pharmaceutical composition.

It is further contemplated that pharmaceutical compositions as described herein can synergize with additional therapeutic agents. For example, it is contemplated that for tumors comprising inflamed portions permeated by T cells with low expression of P3028 can be amenable to treatment with check point inhibitors (See Example 7). For example, it is contemplated that antibodies against PD-1 or PD-L1 can inhibit PD-L1-mediated of immunosuppression, and thus can synergize with peptide inhibitors as described herein. As such, in some embodiments, the method further comprises administering an additional therapeutic agent to the patient. In some embodiments, the additional therapeutic agents comprises, consists essentially of, or consists of an antibody specific for PD-1, and/or an antibody specific for PD-L1. In some embodiments, the additional therapeutic agent is an antibody specific for PD-L1, selected from the group consisting of durvalumab, atezolizumab and avelumab. In some embodiments, the additional therapeutic agent is an antibody specific for PD-1, selected from the group consisting of nivolumab, and pembrolizumab. In some embodiments, the additional therapeutic agent is an antibody or antibody fragment that is bispecific for PD-1 and PDL-1

### Example 1: Effect of a Low Molecular Weight Inhibitor of P3028 on Lymphocyte Activation

Analyses of the inhibitor of P3028, P28R, were performed in human ex vivo models. The stimulatory activity on PBMCs, measured using the MTS or CFSE techniques, were studied in 7 healthy control samples and 7 cancer patients of various diagnoses. Interestingly, even in the absence of other types of stimulation P28R has a significant stimulatory activity in 6 out of 7 cancer patients whereas PBMCs from control samples showed only a weak or no stimulation.

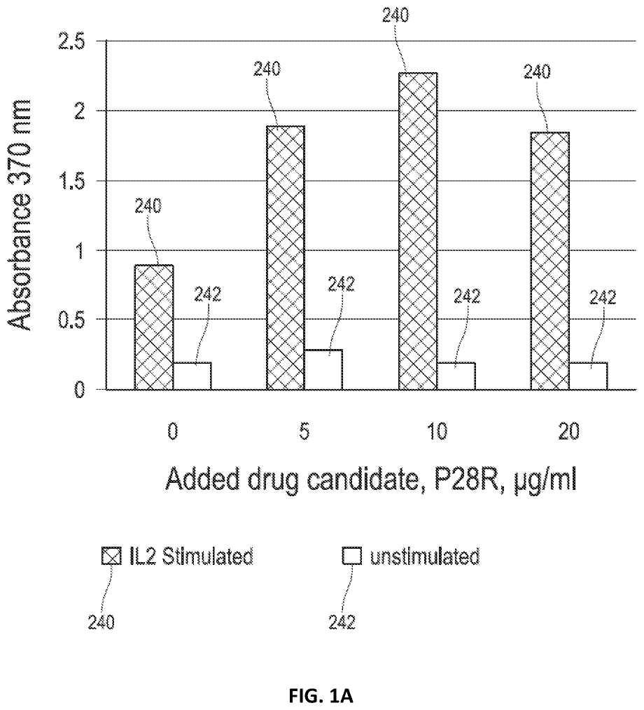

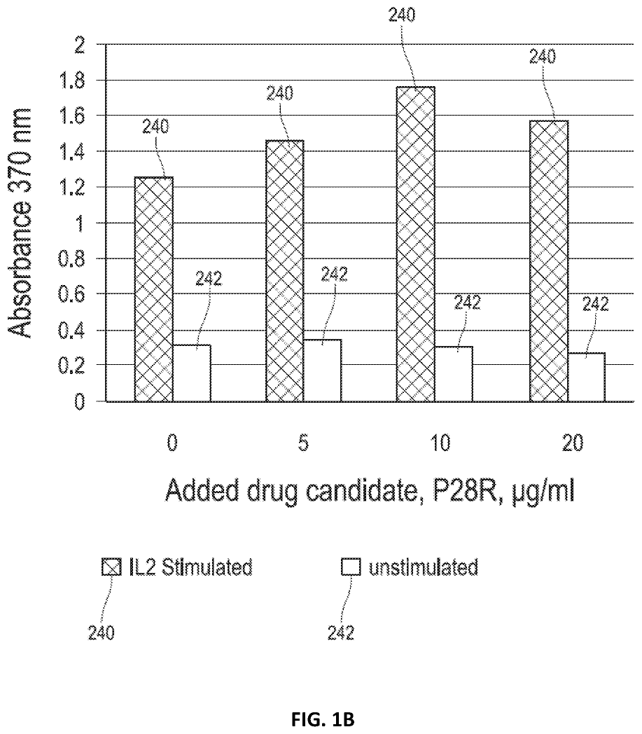

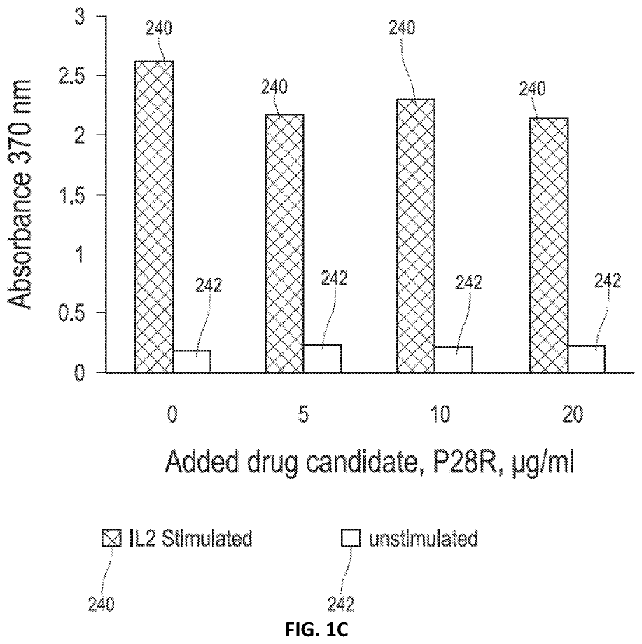

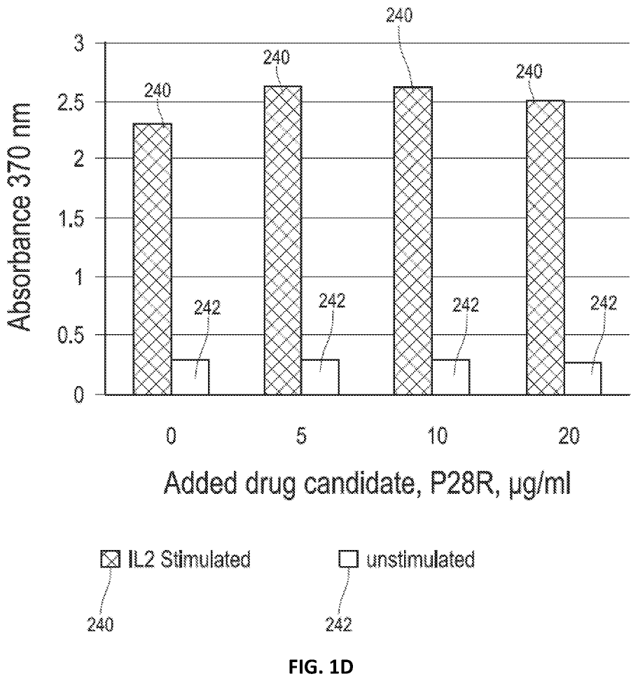

As shown in FIGS. 1A-D, stimulatory activity of P28R on suppressed proliferative response to IL-2. PBMCs were cultured for 7 days with IL-2 and the proliferative rate was determined as incorporation of BrdU. Each bar represents mean value of triplets. Similar to the studies on the efficacy of antibodies (see FIG. 22 of PCT Pub. No. WO 2016/144650) directed against P3028 to reverse cancer related immunosuppression determined as a poor proliferative response of PBMCs from cancer patients to IL-2, the efficacy of the low molecular weight inhibitor P28R on reversal of suppressed IL-2 induced proliferation was investigated. The results of cultures of PBMCs from four different treatment naïve patients are shown in FIGS. 1A-D. For each quantity of added P28R, IL-2 stimulated cells 240 are shown in the left, and unstimulated 242 are shown on the right. PBMCs with a low initial proliferation (see FIGS. 1A and 1B) were markedly stimulated by P28R whereas a high initial proliferation was essentially unaffected by the drug (see FIGS. 1C and 1D). As expected, systemic immunosuppression was not present in all patients and only those with immunosuppression were stimulated.

### Example 2: Effect of P28R and P28 Core Peptide on PBMC Activation

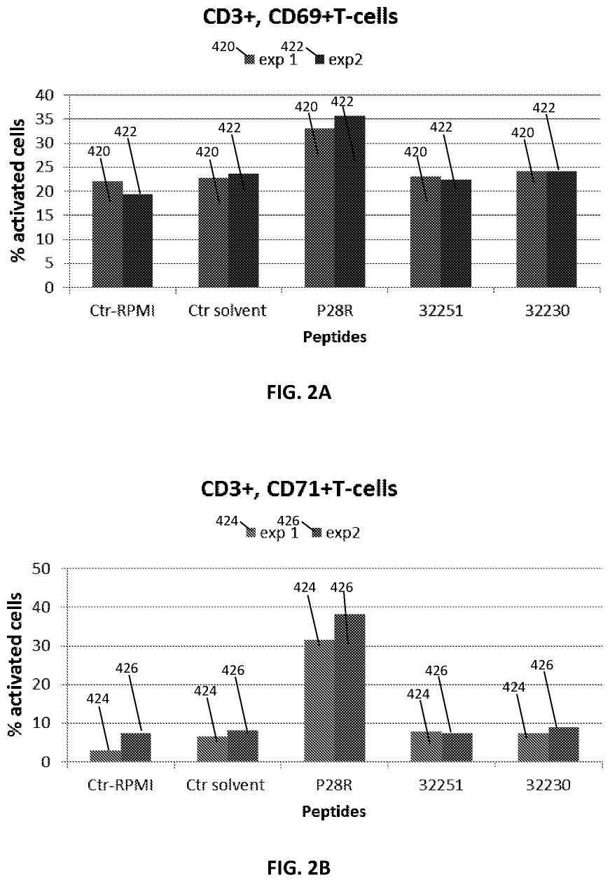

P28R (SEQ ID NO: 2) can stimulate PBMC's from healthy controls in short term cultures when RPMI plus 10% normal human AB serum is used as culture medium. Truncations of P28R were also assessed for their ability to activate PBMC's. PBMCs were incubated with the peptides (40 μg/mL) for 24 hours in RPMI plus 10% human AB serum. PBMC activation was measured as percent cells with enhanced expression of either CD69 (FIG. 2A) or CD71 (FIG. 2B) using flow cytometry. Two experiments were performed for each peptide.

A test for direct stimulatory effects on immune cells was performed. As shown in FIGS. 2A and 2B, peptide P28R (SEQ ID NO: 2) effectively activated healthy PBMC's in this model, but peptide 32251 (SEQ ID NO: 4) and peptide 32230 (“P28 core”)(FFVKLS)(SEQ ID NO: 1) did not activate healthy PBMC's in this model.

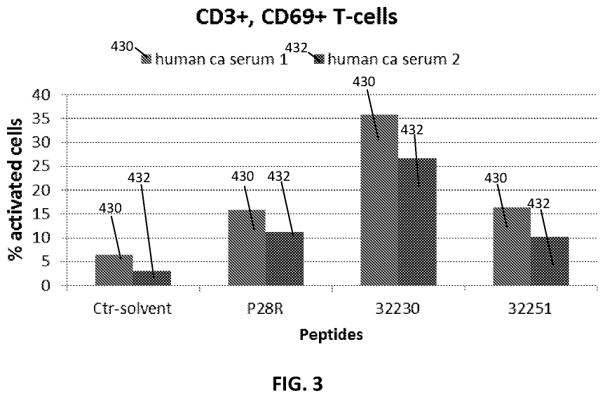

Additionally, P28R and P28 core were tested for their ability to alleviate immunosuppression in cancer cells. In PBMC cultures where normal human AB-serum in the culture medium was substituted for by sera from dogs with cancer or human patients with cancer, P28R (SEQ ID NO: 2) and P28 core (peptide 32230(FFVKLS)(SEQ ID NO: 1) each activated PBMCs, measured as enhanced expression of CD69 (see FIG. 3). FIG. 3 shows a comparison between the full length peptide P28R (SEQ ID NO: 2) and the 6 amino acid P28 core sequence (peptide 32230)(FFVKLS)(SEQ ID NO: 1) in culture medium containing sera from two different cancer patients (human ca serum 1 430 and human ca serum 2 432). Both P28R (SEQ ID NO: 2) and P28 core (SEQ ID NO: 1) activated PBMCs in the presence of cancer serum.

In addition, biotinylated P28R has been shown to bind directly to PBMCs as demonstrated by immunocytochemistry or rosetting of P28R coated beads (binding of beads to the cells).

Taken together, these results show that P28R (SEQ ID NO: 2) can bind to P3028 and de-block cellular receptors and can also have a direct stimulatory activity on immune cells. Additionally, P28 core (SEQ ID NO: 1) can bind to P3028 and de-block cellular receptors.

### Example 3: Activation of the Immune System by P28R in Immunocompetent Mice

The capacity of P28R to activate the immune system and thereby induce tumor cell-lysis was studied in immunocompetent mice, C57B1, with inoculated B16 melanoma. P28R, 20 nM in 100 microliter, was injected intra-tumorally and the tumors were taken out after 3-5 days. As shown in PCT Pub. No. WO 2016/144 (incorporated by reference in its entirety herein) at Example 49, a regional lymph node reaction was regularly found in animals treated intra-tumorally with P28R Intra-tumoral injection of P28R resulted in remarkable tumour regressive changes not only in P28R treated tumours but also in uninjected contralateral tumour (PCT Pub. No. WO 2016/144 at FIGS. 55A-D) or injected with saline only. The effect in the untreated distant/contralateral tumours increased with time after injection of P28R into the treated tumour. Similar results were obtained in a Lewis lung carcinoma model in B57B1 mice. Accordingly, it is shown that administration of peptide inhibitor P28R in accordance with some embodiments herein can induce regressive changes in tumors, including tumors that receive the peptide inhibitor intratumorally, as well as tumors in other parts of the patient (e.g. tumors contralateral to the tumor that received the peptide inhibitor).

### Example 4: Intratumoral Treatment of Spontaneous Tumors in Dogs with P28R

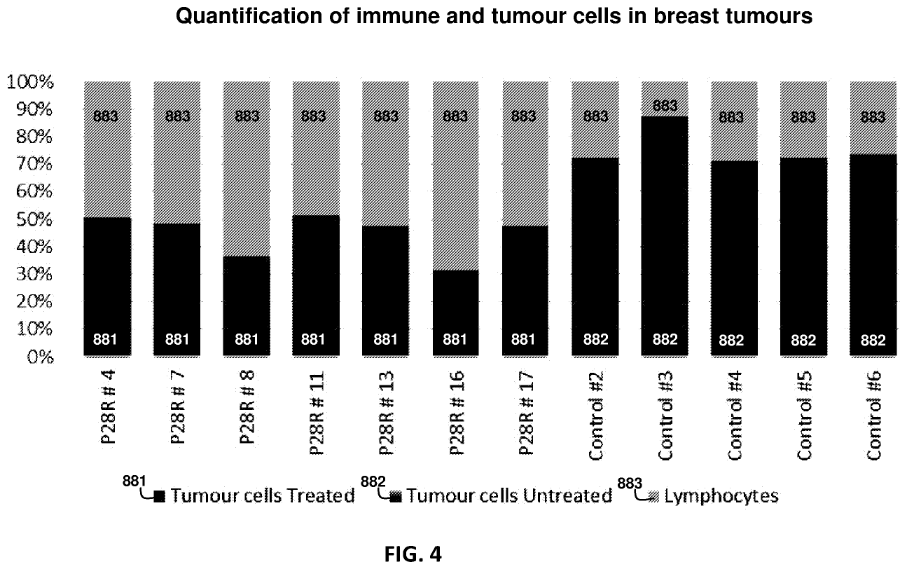

Spontaneous canine tumours, of variable histology have been treated by intra-tumoral injection of 40 nmol P28R in 200 microliters. Treatment of dog tumors with P28R is described in detail in Examples 50-60 of PCT Pub. No. WO 2016/144650, which is incorporated by reference in its entirety herein. Example results quantifying tumor cells (881 for treated dogs, 882 for untreated control dogs) and lymphocytes (883 for both treated dogs and untreated control dogs) 7 dogs with treated mammary tumours and 5 untreated control dogs are shown in FIG. 4. In these dogs with mammary tumours, a marked inflammatory infiltrate was found in all treated tumours and the number of degenerative tumour cells increased compared to 14 untreated tumours. The anti-tumour effect was observed throughout large tumours even if the drug was injected in only 200 μL. In two dogs with multiple tumours the same response to P28R was found not only in the injected tumours but also in uninjected tumours.

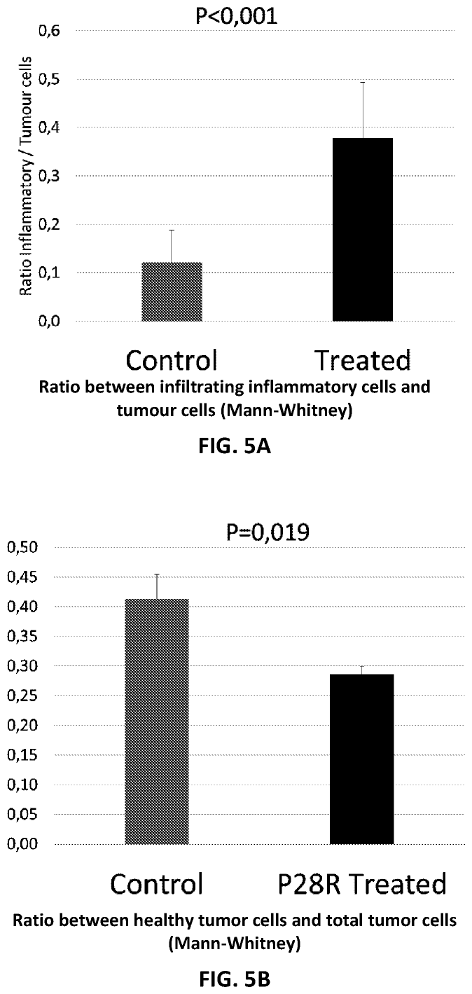

Spontaneous tumours in dogs were treated in a single dose escalation study using subcutaneous injection of P28R and the tumours were resected after 3-5 days. As shown in FIGS. 5A-B, subcutaneous P28R treatment (single dose 80 microgram) of spontaneous canine breast tumours results in recruitment of inflammatory cells and tumour cell death. (Control N=12 and treated N=5). Dog were also dosed at 160 micrograms and 320 micrograms. No adverse events have been observed at any dose level in any dog. Additionally, (A) in a dog with generalised high grade B-cell lymphoma with multiple lymph node enlargements, one injection of P28R at 200 micrograms in 500 microliter was administered into one popliteal lymph node, 2 cm in diameter. 5 days, the injected tumour could not be found clinically. (B) In a dog with progressive squamous cell carcinoma of the alveolar ridge on the right side extending into the hard palate/palatum durum, and up under the right eye with protrusion beneath and lateral to the eye, tumour size 3×4 cm, ten weekly intra-tumoural injections of 80 micrograms of P28R in 200 microliter were administered. After three months, a histopathological complete remission of the injected tumour was achieved. (C) In a dog with multiple, recurrent mammary tumours up to 4×4 mm in size, after 9 usually weekly injections of 80 micrograms in 20 microliter, histopathological examination shows a strong inflammatory response, but also remaining tumour cells. In summary, it can be concluded the peptide inhibitor P28R of some embodiments is well-tolerated, and effective in ameliorating, inhibiting, and treating multiple types of tumours.

### Example 5: Systemic Effect of P28R

A study on the effect of systemic, subcutaneous administration of P28R was performed. This study is described in Example 62 of PCT Pub. No. WO 2016/144650. Mice with inoculate CT26 colon cancer were treated with 12 microgram P28R, twice weekly for two weeks. Apoptosis was induced in the majority of tumour cells. These results show that systemic administration, in addition to direct tumoral injection, of peptide inhibitors such as P28R in accordance with some embodiments herein can induce an inflammatory infiltration, tumor cell regressive changes, and/or eradication of tumor tissues throughout the patient, and can induce programmed cell death in the tumor cells.

### Example 6: Synergy Between P28R and Antibodies Against PD-1 and PD-L1

In the development of the cancer drug P28R an antibody directed against CD45 was chosen just in order to evaluate an enhanced inflammatory response. This marker does not allow differentiation between the functional characteristics of different subsets of intra-tumoral inflammatory subsets. In a large number of the studies the short-term period after injection of P28R was investigated in particular in the dog studies, that is mainly activation of the innate immune system. In the mouse experiments, a strong inflammatory response was induced within 3-5 days, in some experiments already after 24 hours. Interestingly, in this short time period an impressive eradication of tumour cells was observed. Evaluation of tumour cell death was based on morphological criteria, TUNEL and Feulgen staining. It was obvious that a strong anti-tumour activity was elicited. In addition, a statistically significant synergistic effect between P28R and antibodies directed against PD-1 and PD-L1 was demonstrated, evaluated as growth retardation. Without being limited by theory, in view of this observation, the focus on dissecting the inflammatory response was more directed on anti-tumour effector mechanism than on the possible occurrence of suppressor mechanisms. Furthermore, without being limited by theory, it could be concluded that macrophages were the main type of effector cells, based on the early response, the morphology of the dominating inflammatory cells and macrophage specific markers such as F4/80. A significantly enhanced recruitment of CD8+ cells occurred after a prolonged treatment period of 2 weeks, which is in good agreement with an early innate induction of immunogenic cell death follow by an adaptive immune response.

### Example 7: Presence of P3028 Structures in Human Cancer

Currently, therapeutic hurdles in immunotherapy of cancer can be described by the immune blockade phenotypes: immune desert tumours (no infiltration by T-cells), immune excluded tumours (T-cells restricted to surrounding stromal areas) and inflamed tumours (infiltration by non-functional T-cells). The occurrence of the immunosuppressor 3028 correlates with poor recruitment of T-cells to tumours as shown in studies breast cancer and head and neck cancer. This is in good agreement with 3028 blocking of LFA-1 and thereby inhibiting lymphocytes capacity to enter tumours and within tumours migrate closed to tumour cells.

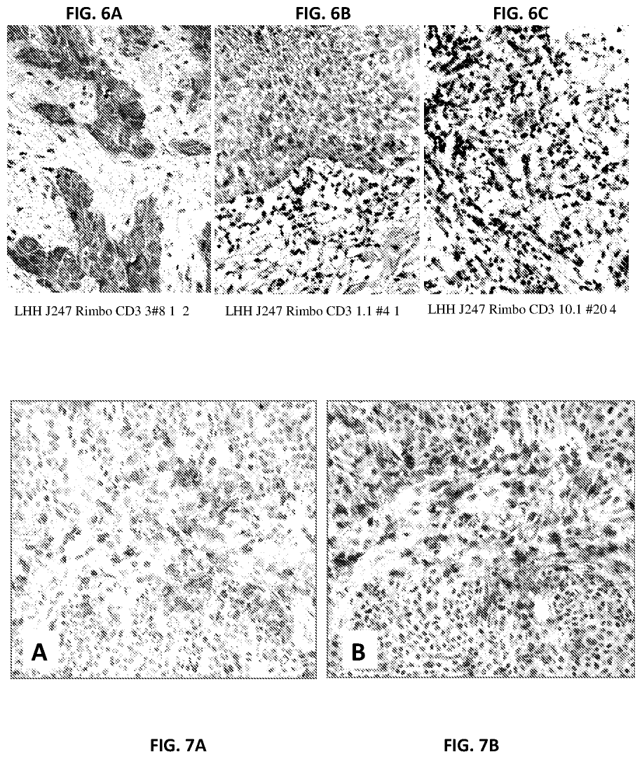

Three different tongue cancers double stained using antibodies directed against 3028 (red) and CD3 (brown) are shown. The immune desert cancer to the left (FIG. 6A) has a strong expression of 3028 and only few scattered T-cells in the stroma, the immune excluded cancer in the middle (FIG. 6B) has a strong expression of 3028 and T-cells infiltrating in the stroma, the inflamed cancer to the right (FIG. 6C) is only faintly stained for 3028 and has a very strong infiltration of T-cells.

Without being limited by theory, it is contemplated that conventional immunotherapy may be challenging in cancers with the immune blockade shown in FIGS. 6A and 6B. Without being limited by theory, in cancers characterized as inflamed, there might still to a large extent be migration blockade, due to LFA-1 blockade, inhibiting migration of T-cells close to the tumour cells. It is further contemplated that these types of cancers are likely to respond to pharmaceutical formulations comprising peptide inhibitors as described herein. In the type of inflamed cancer shown in FIG. 6C, which is permeated by T-cells and have a low expression of 3028 other suppressor mechanisms such as check point molecules are operating. These are the tumours most likely to respond to check point inhibitors.

### Further Investigation on Human Head and Neck Cancer

The expression of the immunosuppressor 3028 in cancers of the oral cavity was studied. Out of 18 evaluated tongue, larynx and tonsil samples 16 showed medium to high expression of 3028 in the tumor nodules. Also the two remaining samples showed expression of 3028 but to a lesser extent.

Statistical analysis showed that the presence of CD3+ tumor infiltrating lymphocytes (TIL) correlated with a p-value of 0.024 indicating that the amount of CD3+ lymphocytes significantly correlates with the presence of 3028 in tumor nodules.

### Example 8: Ex Vivo Treatment of Human Breast Cancer

To further demonstrate the biological relevance of LFA-1 blocking by 3028-expressing albumin structures in tumours, fresh frozen tumour sections from a human breast cancer were incubated with P28R before staining for LFA-1 (CD11a). For comparison, tumour sections were incubated with phosphate buffered saline only. P28R reversed the blocking of LFA-1 leading to enhanced intensity of staining and likely also enhanced function of the LFA-1 receptor that is important for lymphocyte migration and cytotoxic activity (FIGS. 7A-B).

FIGS. 7A-B show tumour sections from a breast cancer patient showing inflammatory cells stained by an antibody directed against CD11a. Fresh frozen tumour sections without any fixation were incubated with buffer (FIG. 7A) or P28R (FIG. 7B) before staining. Inflammatory cells are observed infiltrating the tumor section treated with P28R.

In addition, sections from human breast and tongue cancers were treated ex vivo with P28R. In cancers with a strong spontaneous inflammatory response close to tumour cells, immune cells displayed a strong membrane staining of LFA-1, whereas in tumours with only moderate infiltration of inflammatory cells the membrane staining was blocked but could be un-blocked by ex vivo P28R treatment.

### Example 9: Preparation of “Formulation A” and “Formulation B”

P28R is a short peptide with a propensity to form P-sheets in neutral pH. It has been successfully formulated in an acetate buffer with pH 5.2 (“Formula A”; See Table 2A) and used as vehicle for P28R in a study on dogs with spontaneous tumors (study CIG-1301)(See Example 4; See also PCT Pub. No. WO 2016/144650). When analyzing the effect of P28R after intratumoral administration, a clear treatment effect could be detected, but cells close to the injection site tumors looked slightly affected by the injected vehicle. Without being limited by theory, this was thought to originate from the acetate and/or the low osmolality of Formula A. Therefore, the acetate levels were drastically reduced and the osmolality adjusted to an isotonic level by addition of sodium chloride to produce “Formula B” (See Table 2B).

TABLE 2A
Formula A

| 0.4 mg/mL P28R in Formula A contains the following ingredients: | |
|---|---|
| CS8040 (P28R) | 0.20 mM |
| Acetic Acid | 34.8 mM |
| Sodium Acetate | 43.3 mM |
| Sodium Hydroxide | 20.0 mM |
| Osmolality: | 141.6 mOsmol/L |

TABLE 2B
Formula B
0.4 mg/mL P28R in Formula B contains the following ingredients:

| | |
|---|---|
| CS8040 (P28R) | 0.20 mM |
| Acetic Acid | 32.8 mM |
| Sodium Acetate | 1.48 mM |
| Sodium Hydroxide | 28.3 mM |
| Sodium Chloride | 112.6 mM |
| Osmolality: | 289.9 mOsmol/L |

P28R in Formula B has, for production technical reasons been divided into two vials that before usage should be mixed to produce P28R in Formula B (See Example 10).

P28R was produced by C S Bio Co. CA, USA according to GMP standards. The chemicals used for vehicle production were acetic acid glacial (Fisher Scientific, UK), sodium chloride (S3014-5 kg, Sigma, Steinheim, Germany), Sodium Hydroxide (Sigma, 221465-500G, Sigma, Steinheim, Germany), sodium acetate (71183-250G, Sigma, Steinheim, Germany). Sterile filters used were: The 25 mm Sterile syringe filter w/ 0.2 μm Polyethersulfone (PES) membrane (European Cat. No. 514-0073 VWR International, Radnor, Pa., USA.) or the 50 mm Baxter 0.2 Micron Filter (REF H93835, Baxter, Deerfield, Ill., USA). Protein concentration was determined using the “peptide” setting in a DeNovix DS-11 spectrophotometer with correlating blank as control. The pH was measured using newly calibrated WTW Inolab pH 720.

### Example 10: The Propensity of P28R to Form Gel is Sodium Hydroxide and/or pH Dependent

It was observed that P28R in Formula B cannot be sterile filtered due to clogging of the filter and the limited amount of flow through that can pass through, has nearly no P28R content (Data not shown). Without being limited by theory, the clogging of the filter is suspected to originate from the possibility of short peptides such as P28R forming pi-sheets and/or gel. P28R peptides in this state form a clear transparent liquid that, when microscopically investigated, is in the form of a very thin gel.

Five different batches of Formula B were produced with different amounts of Sodium Hydroxide added to create a gradient of pH. Small beads were added to the solutions to visualize the presence of any gel formation (Table 3).

Table 3: Gel forming capabilities of Formula B at different pH. The propensity of P28R to form gel in formulations with different pH ranging from pH 5.35 up to 10.97 was identified using addition of small beads. (*Precipitated peptide has a propensity to stick on tube walls thus giving the varied concentration.)

TABLE 3

| NaOH | pH | mg/ml | State | Comment |
|---|---|---|---|---|
| 90% | 5.35 | 0.33 | gel | Detectable |
| 95% | 5.56 | 0.33 | gel | Detectable |
| 100% | 5.87 | 0.33 | gel | Easy to detect |
| 105% | 9.98 | 0.11* | N/A | Precipitates |
| 110% | 10.97 | 0.31* | N/A | Precipitates |

It is observed that the gel formation is affected by addition of sodium hydroxide. Without being limited by theory, the gel formation could be attributable to the sodium addition and/or could be pH dependent. There is, however, an upper pH threshold around pH 10, where gel is not formed, but the formula shifts from a clear solution to a white grainy appearance.

Thus, it is contemplated that the presence of sodium, and/or pH's of at least 5.35 can cause P28R peptide to form a gel, which can interfere with sterile filtration. On the other hand, at lower pH's such as 3.6, the P28R peptide remained dissolved in liquid solution (See, e.g., Table 4).

### Example 11: Sodium Chloride Induces Aggregation of P28R

When sodium hydroxide was added to Formula B, sterile filtering was not possible due to clogging of the filter, but in the absence of both sodium hydroxide and sodium chloride, filtration was possible and no gel formation could be detected (data not shown).

To evaluate if dissolved peptide binds to the membrane during filtration, the Formula B without NaOH and NaCl, resulting in pH 3.6 (10 mg of P28R (CS8040), 471.7 μl of 10% acetic acid and 23.16 ml of 1.6 mM sodium acetate resulting 0.27 mg/ml P28R when measured with a DeNovix DS-11 spectrophotometer) was sterilized through a 0.2 μm PES filter and the flow through aliquoted in 1 ml volumes. P28R concentration of flow through was determined using a DeNovix DS-11 spectrophotometer (Table 4). It is noted that the P28R had an average molecular mass of 1972.3 Da.

Table 4 shows P28R concentration in flow through after sterilizing using a 25 mm PES filter. Formula B without NaOH and NaCl, resulting in pH 3.6 (10 mg P28R (CS8040), 471.7 μl 10% acetic acid and 23.16 ml 1.6 mM sodium acetate) were sterilized trough a 0.2 μm PES filter. P28R concentration of flow through was determined using a DeNovix DS-11 spectrophotometer. (*A limited selection of the samples was measured and the P28R concentration were always between 0.26-0.28 mg/ml, data not shown).

TABLE 4

| Aliquot | mg/ml |
|---|---|
| 1. | 0.26 |
| 2. | 0.28 |
| 3. | 0.28 |
| 4. | 0.26 |
| 5. | 0.27 |
| 6. | 0.28 |
| 7. | * |
| 8. | * |
| 9. | * |
| 10. | * |
| 11. | * |
| 12. | * |
| 13. | * |
| 14. | * |
| 15. | * |
| 16. | * |
| 17. | * |
| 18. | * |
| 19. | * |
| 20. | * |
| 21. | * |
| 22. | * |
| 23. | * |
| 24. | 0.27 |

The same batches were then filtered through the 50 mm Baxter filter from Pharma-Skan and the protein content in the flow-through was determined spectrophotometrically (Table 5).

Table 5 shows P28R concentration in flow through after sterilizing using a 50 mm Baxter filter. Formula B without NaOH and NaCl was sterilized through a 50 mm Baxter filter and the flow through aliquoted in 1 ml volumes. P28R concentration of the first 24 milliliters of flow through was determine using a DeNovix DS-11 spectrophotometer. (* The liquid filled a previously dry area on the membrane during this time).

TABLE 5

| Aliquot | mg/ml |
|---|---|
| 1. | 0.18 |
| 2. | 0.24 |
| 3. | 0.26 |
| 4. | 0.26 |
| 5. | 0.26 |
| 6. | 0.27 |
| 7. | 0.27 |
| 8. | 0.27 |
| 9. | 0.27 |
| 10. | 0.27 |
| 11. | 0.27 |
| 12. | 0.27 |
| 13. | 0.25* |
| 14. | 0.26 |
| 15. | 0.26 |
| 16. | 0.27 |
| 17. | 0.27 |
| 18. | 0.28 |
| 19. | 0.27 |
| 20. | 0.28 |
| 21. | 0.27 |
| 22. | 0.27 |
| 23. | 0.27 |
| 24. | 0.27 |

For further evaluation of the impact of pH on P28R behavior in formulated state, the samples in tubes 3-12 and 15-25 were pooled and the pH adjusted to 4.8 using sodium hydroxide, the protein concentration determined to 0.26 mg/ml before the solution was ran through the same Baxter filter as used in Table 5. The protein content was determined for the six first milliliters of flow-through (1.0 ml/tube) (Table 6).

Table 6 shows that elevated pH induces P28R binding to 50 mm Baxter filter. Filtration of Formula B with pH 4.8 without NaCl through a filter saturated with P28R from previous filtration of Formula B with pH 3.6. P28R concentration of the six first milliliters of flow through was determined using a DeNovix DS-11 spectrophotometer.

TABLE 6

| Aliquot | mg/ml |
|---|---|
| 1. | 0.22 |
| 2. | 0.25 |
| 3. | 0.26 |
| 4. | 0.25 |
| 5. | 0.26 |
| 6. | 0.26 |

That the first ml of flow through contains a slightly lower concentration than the following samples indicates that the increased pH (of 4.8) induces the peptide to bind to the filter (compared to the pH of 3.6). The filter should already be saturated from the filtration done in the experiment displayed in Table 5, but the elevated pH might change either the filter properties or the peptide in the formula to facilitate the increased P28R binding capability.

To investigate if sterile filtration of a higher P28R concentration is possible in Formula B without NaCl and NaOH, a new batch with a protein content of 0.37 mg/ml was produced and filtered through a PES filter (Table 7).

Table 7 shows P28R concentration in flow through after sterilizing using a PES filter. Filtration of Formula B with pH 3.6 and a P28R content of 0.37 mg/ml. P28R concentration of the 12 first milliliters of flow through were determined using a DeNovix DS-11 spectrophotometer.

TABLE 7

| Aliquot | mg/ml |
|---|---|
| 1. | 0.31 |
| 2. | 0.34 |
| 3. | 0.36 |
| 4. | 0.36 |
| 5. | 0.37 |
| 6. | 0.34 |
| 7. | 0.36 |
| 8. | 0.36 |
| 9. | 0.36 |
| 10. | 0.36 |
| 11. | 0.35 |
| 12. | 0.37 |

The capability of P28R to form gel is observed when suspended in Formula B but not in Formula A. Without being limited by theory, this phenomenon is contemplated to be the most probable cause behind the possibility to sterile filter Formula A without clogging but not Formula B. Due to the high acetate concentration and low osmolality of Formula A, Formula B is contemplated to be more suitable for clinical use.

To investigate the reason behind gel formation, several versions of Formula B with lower pH and/or without NaCl were investigated. Formulations containing NaCl could not be passed through a sterile filter, demonstrating that NaCl or the resulting elevation of osmolality is inducing gel formation of P28R peptides.

After filtration of Formula B without NaCl, it was evident that a small proportion of P28R could bind to the filter, before saturation was reached. The amount of P28R that could bind to the filter before saturation was pH dependent as shown in Table 5.

The amount of P28R that can bind to the filter is dependent on both the material type and the filter size. The total amount of peptide needed to saturate the 50 mm Baxter filter is in the magnitude of 0.1 mg P28R, calculated from the loss of peptide after filtration in Table 6. This Baxter filter is identical to the filters used by Pharma-Skan ApS during sterilization of P28R made for clinical use. Most often, more than 100 mg of P28R is used in the production of a clinical batch and a loss of 0.1 mg equals to less than 1/1000 is thus considered neglectable.

### Example 12: Formulation of P28R for Clinical Use in Accordance to GMP

Production of Formula B using a two-vial in accordance with current GMP standards was done by Pharma-Skan ApS in batch No. 611170 (Using API CS8040 batch GH1008). The P28R concentrate (P28R Vial-A) contained all P28R in 0.76 mL of Formula B buffer without the NaCl and NaOH. The diluent (P28R Vial-B) contained all the NaCl and NaCH in a 0.24 mL solution. Fusing of “P28R Vial-A” and “P28R Vial-B” to form Formula B was later done in the clinic right before injection.

Formula B is isotonic and has less acetate compare with Formula A, making Formula B preferential for clinical usage. The gel inducing capability of Formula B, due to NaCl content, is causing a production technical problem during the filtration sterilization step. To circumvent this issue, P28R can be sterile filtered in the absent of NaCl and at low pH. Therefore, production of P28R for clinical use can be accomplished using a division of Formula B into a two-vial system (The P28R concentrate in “P28R Vial-A” and the diluent P28R Vial-B”). Vial-A will consist of P28R dissolved in acetic acid and sodium acetate. Vial-B will contain sodium chloride and sodium hydroxide. To avoid exerting P28R for elevated pH during blending of the two vials, Vial-B, sodium hydroxide, must be added into Vial-A containing the P28R. When mixing Vial A and Vial B, Formula B will be produced, ready for clinical use.

### Example 13: Stability Testing of GMP Produced P28R Batch

The stability of batch 611170 was tested in collaboration with BirkaBioStorage AB for monitored storage conditions and HPLC purity analysis performed by Q&Q-Labs AB. Purity analysis was done immediately before storage and the result 97.0% pure was used as a reference value to compare for later analyses. After 19 months storage in +5° C. the purity was down to 94.8%. All stability results are presented in Table. 8.

Table 8 shows P28R stability results from batch 611170. P28R concentrate (P28R Vial-B) stored in a monitored +5° C. condition by the certified storage facility Birka Biostorage AB (Lund, Sweden) was purity tested using HPLC at indicated time points by Q & Q—Labs AB (Mölndal, Sweden) in compliance with current GMP standard. The purity of P28R peptide, largest individual related and additional individual related substances above 0.1% are presented in the table.

TABLE 8

| | | Largest | |
|---|---|---|---|
| | | individual | |
| Time point | Purity | related | Additional individual related |
| (months) | (%) | (%) | >0.1% |
| Initial | 97.0% | 0.8% | 0.4% 0.3%, 0.3%, 0.2%, 0.2% |
| 3 | 96.1% | 0.8% | 0.8%, 0.5%, 0.3%, 0.3%, 0.3%, 0.2% |
| 8 | 96.4% | 1.9% | 0.4%, 0.4%, 0.3%, 0.2% |
| 9 | 96.0% | 2.0% | 0.3%, 0,3%, 0.3%, 0.2%, 0.2%, 0.2%, |
| | | | 0.2% |
| 11 | 95.3% | 1.4% | 1.3%, 0.4%, 0.4%, 0.3%, 0.3% |
| 12 | 94.9% | 1.4% | 1.3%, 0.5%, 0.4%, 0.3%, 0.3%, 0.2%, |
| | | | 0.2% |
| 14 | 94.6% | 1.5% | 1.4%, 0.5%, 0.4%, 0.3%, 0.3%, 0.3%, |
| | | | 0.2% |
| 19 | 94.8% | 2.0% | 1.5%, 0.6%, 0.3%, 0.2% |

### Example 14: Vehicle for High Concentration P28R

For future use, an optimized “Formulation C” with the possibility to increase P28R concentration significantly was developed.

Formula C is produced by dissolving P28R in 0.01M Acetic Acid to a concentration of 13.3 mg/ml to which 1.12M Glucose is added to give an isotonic solution with a P28R concentration of 10 mg/mL. The acidity of the 0.01M Acetic Acid is counteracted by the peptide counter ion, which is acetate, thus giving a pH around 5.2. This Formula C 10 mg/mL can be sterile filtered without clogging the filter, but is viscose and bubbles significantly. The important part is that it does not form gel and is therefore an alternative if high P28R concentrations will be needed in the future. Formula C with lower P28R concentrations can easily be produced by diluting with isotonic Glucose. Preliminary investigation of Formula C with P28R indicate that concentrations below 5 mg/mL are practical to utilize.

Formula C has high potential in several regards including costs of production, production technical, mild buffer capacity, possibility to increase P28R concentration and it is more practical to have a single-vial system rather than a two-vial system in the clinic.

In at least some of the above-described embodiments, one or more elements used in an embodiment can interchangeably be used in another embodiment unless such a replacement is not technically feasible. It will be appreciated by those skilled in the art that various other omissions, additions and modifications may be made to the methods, compositions, pharmaceutical products, pharmaceutical formulation, and uses described herein without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a device or substrate having at least one of A, B, and C” would include but not be limited to devices or substrates that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a device or substrate having at least one of A, B, or C” would include but not be limited to devices or substrates that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.” Additionally, when a method comprising a composition, pharmaceutical formulation, or pharmaceutical product is described herein, the corresponding composition, pharmaceutical formulation, or pharmaceutical product for use is also contemplated. For example, a method of treating cancer comprising administering a pharmaceutical formulation comprising a peptide inhibitor, expressly also contemplates a pharmaceutical formulation comprising a peptide inhibitor for use in treating cancer.

In addition, where features or aspects of the disclosure are described in terms of Markush groups, those skilled in the art will recognize that the disclosure is also thereby described in terms of any individual member or subgroup of members of the Markush group.

As will be understood by one of skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those of skill in the art. The various aspects and embodiments disclosed herein are for purposes of illustration and are not intended to be limiting, with the true scope and spirit being indicated by the following claims.

1. A pharmaceutical product comprising:
a first solution comprising an isolated peptide comprising amino acid sequence FFVKLS (SEQ ID NO: 1) dissolved in the first solution, the first solution having a pH less than 7 or about less than 7, and a sub-isotonic osmolarity; and
a second solution comprising a tonicity agent and a base,
wherein the first and second solution generate an isotonic gel when combined with each other, wherein the isotonic gel comprises the isolated peptide at a concentration of at least 0.2 mg/ml or about at least 0.2 mg/ml and a pH of 6.5-7.5 or about 6.5-7.5.

2-61. (canceled)
